(19) United States
(12) Patent Application Publication
(10) Pub. No.: US 2009/0131384 A1
(43) Pub. Date: May 21, 2009

(54) COMPOUNDS AND METHODS FOR TREATMENT OF DISORDERS ASSOCIATED WITH ER STRESS

(75) Inventors: Teoman Uysal, Brookline, MA (US); Nicholas D.P. Cosford, San Diego, CA (US)

Correspondence Address: EDWARDS ANGELL PALMER & DODGE LLP, P.O. BOX 55874, BOSTON, MA, 02205, US

(73) Assignee: SYNDEXA PHARMACEUTICALS CORPORATION, Wellesley, MA (US)

(21) Appl. No.: 12/294,029

(22) PCT Filed: Mar. 22, 2007
(86) PCT No.: PCT/US07/07228

§ 371 Date: Dec. 16, 2008

Related U.S. Application Data
(60) Provisional application No. 60/785,235, filed on Mar. 22, 2006.
(60) Provisional application No. 60/785,182, filed on Mar. 22, 2006.
(60) Provisional application No. 60/785,007, filed on Mar. 22, 2006.
(60) Provisional application No. 60/785,035, filed on Mar. 22, 2006.
(60) Provisional application No. 60/785,185, filed on Mar. 22, 2006.
(60) Provisional application No. 60/785,328, filed on Mar. 22, 2006.
(60) Provisional application No. 60/785,338, filed on Mar. 22, 2006.
(60) Provisional application No. 60/785,335, filed on Mar. 22, 2006.
(60) Provisional application No. 60/785,154, filed on Mar. 22, 2006.
(60) Provisional application No. 60/785,034, filed on Mar. 22, 2006.

(51) Int. Cl. A61K 31/58; C07J 41/00; C07D 239/26; C07C 229/34; A61K 31/426; A61K 31/4409; A61K 31/13; A61P 9/10; A61K 31/195; A61K 31/495; C07D 277/30; A61K 31/575

(52) U.S. Cl. 514/176; 552/554; 514/182; 544/335; 548/204; 562/450; 514/252.12; 514/365; 514/561; 514/277; 514/644

(57) ABSTRACT

The invention provides novel compounds, methods for treating or preventing a condition related to ER-stress, e.g. hypercholesterolemia, atherosclerosis and related conditions, and pharmaceutical compositions related thereto.

### RELATED APPLICATIONS

This application claims the benefit of priority to U.S. Provisional Application Nos. 60/785,328, 60/785,185, 60/785,154, 60/785,035, 60/785,007, 60/785,338, 60/785,182, 60/785,034, 60/785,335 and 60/785,235, all filed on Mar. 22, 2006. This application is related to the subject matter of U.S. patent application Ser. No. 11/227,497, filed Sep. 15, 2005, and U.S. patent application Ser. No. 11/227,543, filed Sep. 15, 2005. The contents of the aforementioned applications are each incorporated herein by reference, in their entireties.

### BACKGROUND OF THE INVENTION

Obesity has been found to be associated with the activation of cellular stress signaling pathways (Uysal et al. Nature 389:610, 1997; Hirosumi et al. Nature 420:333, 2003; Yuan et al. Science 293:1673, 2001; each of which is incorporated herein by reference). One player in the cellular stress response is the endoplasmic reticulum (ER), a membranous network that functions in the synthesis and processing of secretory and membrane proteins. The ER is responsible for the processing and translocation of most secreted and integral membrane proteins of eukaryotic cells. The lumen of the ER provides a specialized environment for the posttranslational modification and folding of these proteins. Properly folded proteins are cleared for exit from the ER and progress down the secretory pathway, while unfolded or misfolded proteins are disposed of by ER-associated protein degradation machinery. The load of proteins that cells process varies considerably depending on the cell type and physiological state of the cell. Cells can adapt by modulating the capacity of their ER to process proteins and the load of protein synthesized, disequilibrium between ER load and folding capacity is referred to as ER stress (Harding et al. Diabetes 51(Supp. 3):S455, 2002; incorporated herein by reference). ER stress has been shown to be triggered by hypoxia, hypoglycemia, exposure to natural toxins that perturb ER function, and a variety of mutations that affect the ability of client proteins to fold (Lee, Trends Biochem. Sci. 26:504-510, 2001; Lee, Curr. Opin. Cell Biol. 4:267-273, 1992; each of which is incorporated herein by reference).

In addition, hypercholesterolemia may be associated with the activation of cellular stress signaling pathways. Hypercholesterolemia is a prevalent and growing health problem throughout the world. Hypercholesterolemia refers to the presence of high or excessive levels of cholesterol in the blood. Hypercholesterolemia can lead to the development of atherosclerotic plaques in arteries and, eventually, to atherosclerosis, stroke, ischemic vascular disease, dyslipidemia and hypercholesterolemia and other complications of these conditions. These cholesterol-associated diseases have become serious threats to human health.

Certain pathological conditions have been shown to disrupt ER homeostasis thereby leading to the accumulation of unfolded and misfolded proteins in the ER lumen (Hampton Curr, Biol. 10:R518, 2000; Mori Cell 101:451, 2000; Harding et al. Annu. Rev. Cell Dev. Biol. 18:575, 2002; each of which is incorporated herein by reference). To cope with ER stress, cells activate a signal transduction system linking the ER lumen with the cytoplasm and nucleus, called the unfolded protein response (UPR) (Hampton Curr. Biol. 10:R518, 2000; Mori Cell 101:451, 2000; Harding et al. Annu. Rev. Cell Dev. Biol. 18:575, 2002; each of which is incorporated herein by reference), Among the conditions that trigger ER stress are glucose and nutrient deprivation, viral infections, increased synthesis of secretory proteins, and the expression of mutant or misfolded proteins (Ma et al., Cell 107:827, 2001; Kaufman et al. Nat. Rev. Mol. Cell. Biol. 3:411, 2002; each of which is incorporated herein by reference).

U.S. patent application Ser. No. 11/227,497, describes certain compounds for reducing ER stress. However, additional compounds and methods for treatment of conditions associated with ER stress are needed.

### SUMMARY OF THE INVENTION

Accordingly, the present invention relates to the use of known and novel compounds for the prevention or treatment of conditions associated with ER stress. For example, in particular embodiments of the invention, the compounds of the invention have been identified for their use in treating or preventing hypercholesterolemia, atherosclerosis, and related conditions. The present invention further relates to methods of identification of compounds that modulate ER stress, pharmaceutical compositions, and packaged formulations.

Accordingly, in one aspect, the invention provides compounds having formula I:

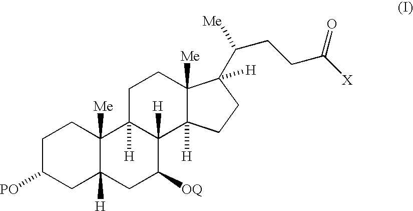

in which
P and Q are independently for each occurrence H, lower alkyl, PO₃R₂, SO₃R or COR;
X is OR, NR₂ or NR—B-D;
B is alkylene, substituted alkylene, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″, or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R is H, lower alkyl, aryl, or heterocyclyl;
R₁-R₄ are independently for each occurrence H, lower alkyl, fluoro or haloalkyl;
R₅ is H, lower alkyl, or substituted or unsubstituted aryl, or substituted or unsubstituted heteroaryl;
m is 1-3; and
n is 0-3;
   and pharmaceutically acceptable esters, salts, and prodrugs thereof. In certain preferred embodiments, the compound of the invention is not tauroursodeoxycholic acid (TUDCA) or a TUDCA analog represented by the formula:

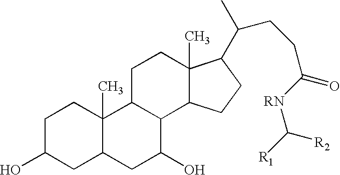

in which:

R is —H or C₁-C₄ alkyl;

R₁ is CH₂—SO₃R₃ and R₂ is —H; or R₁ is —COOH and R₂ is CH₂—CH₂—CONH₂, —CH₂—CONH₂, —CH₂—CH₂—SCH₃ or CH₂—S—CH₂—COOH; and
R3 is —H or a basic amino acid; or a pharmaceutically acceptable salt thereof. In addition, in certain preferred embodiments directed to novel compounds of the present invention, the compound is not one of the following compounds:

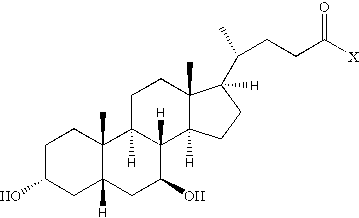

in which X is one of the following moieties:

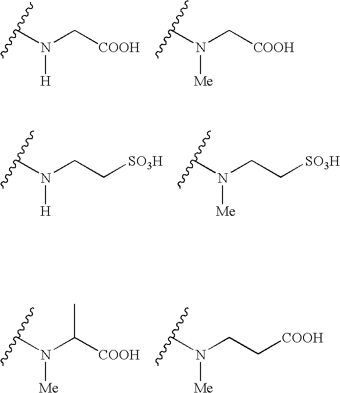

Another aspect of the invention provides compounds represented by Formula II:

A-B-D (II)

wherein:

A is substituted or unsubstituted aryl, or substituted or unsubstituted heteroaryl;

B is alkyl, substituted alkyl, or —(CR₁R₂)ₙ-E-(CR₃R₄)ₘ—;

D is CO₂R, CONRₐR_{b}, SO₃R, SO₂NRₐR_{b}, tetrazolyl, —B(OR)(OR″), —P(O)R′OR″ or PO₃R′R″;

E is O, S, SO, SO₂, —SO₂N(R₅)—, —N(R₅)SO₂—, NR₅, —C(O)O—, —O(O)C—, —CONR₅—, —NR₅CO—, or is absent;

R, R′ and R″ are independently for each occurrence H or lower alkyl;

Rₐ and R_{b} are independently for each occurrence H, OH, lower alkyl, substituted or unsubstituted aryl, or substituted or unsubstituted heteroaryl, provided that Rₐ and R_{b} are not both OH;

R₁-R₄ are independently for each occurrence H, halogen, lower alkyl, or haloalkyl;

R₅ is H, lower alkyl, or substituted or unsubstituted aryl, or substituted or unsubstituted heteroaryl;

m is 1-3; and

n is 0-3;

and pharmaceutically acceptable esters, salts, and prodrugs thereof. In certain preferred embodiments of the invention, the compound is not 4-phenylbutyric acid (PBA), or the compound of Formulas II-IIIb is not represented by the following formula:

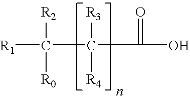

wherein

n is 1 or 2;

R₀ is aryl, heteroaryl, or phenoxy, the aryl and phenoxy being unsubstituted or substituted with, independently, one or more halogen, hydroxy or lower alkyl;

R₁ and R₂ are independently H, lower alkoxy, hydroxy, lower alkyl or halogen; and

R₃ and R₄ are independently H, lower alkyl, lower alkoxy, or halogen; or

a pharmaceutically-acceptable derivative or salt thereof.

In another aspect, the invention provides compounds of Formula H, represented by Formula IIa or IIb:

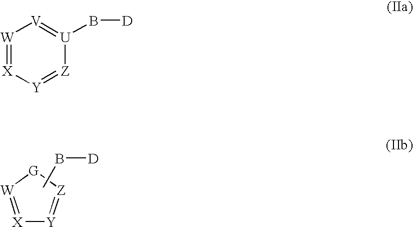

wherein:

U is C or N;

V, W, X, Y, and Z are independently for each occurrence CR₆, NR₇, O, or S;

G is NR₇, O, or S;

R₆ is independently for each occurrence H, halogen, alkyl, aryl, or heterocyclyl;

R₇ is H, alkyl, aryl, or heterocyclyl, or is absent;

B is alkyl, substituted alkyl, or —(CR₁R₂)ₙ-E-(CR₃R₄)ₘ—;

D is CO₂R, CONRₐR_{b}, SO₃R, SO₂NRₐR_{b}, tetrazolyl, —B(OR)(OR″), —P(O)R′OR″ or PO₃R′R″;

E is O, S, SO, SO₂, —SO₂N(R₅)—, —N(R₅)SO₂—, NR₅, —C(O)O—, —O(O)C—, —CONR₅—, —NR₅CO—, or is absent;

R, R′ and R″ are independently for each occurrence H or lower alkyl;

Rₐ and R_{b} are independently for each occurrence H, OH, lower alkyl, substituted or unsubstituted aryl, or substituted or unsubstituted heteroaryl, provided that Rₐ and R_{b} are not both OH;

R₁-R₄ are independently for each occurrence H, halogen, lower alkyl, or haloalkyl;

R₅ is H, lower alkyl, or substituted or unsubstituted aryl, or substituted or unsubstituted heteroaryl;

m is 1-3; and

n is 0-3;

and pharmaceutically acceptable esters, salts, and prodrugs thereof.

In yet another aspect, the invention provides compounds of Formula II, represented by Formula IIIa or IIIb:

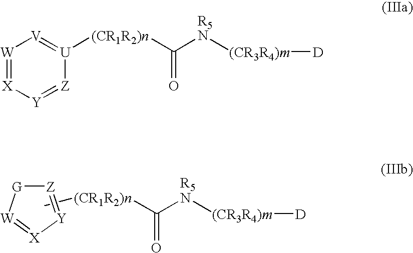

wherein

U is C or N;

V, W, X, Y, and Z are independently for each occurrence CR₆, NR₇, O, or S;

G is NR₇, O, or S;

R₆ is independently for each occurrence H, halogen, alkyl, aryl, or heterocyclyl;

R₇ is H, alkyl, aryl, or heterocyclyl, or is absent;

D is CO₂R, CONRₐR_{b}, SO₃R, SO₂NRₐR_{b}, tetrazolyl, —B(OR)(OR″), —P(O)R′OR″ or PO₃R′R″;

R, R′ and R″ are independently for each occurrence H or lower alkyl;

Rₐ and R_{b} are independently for each occurrence H, OH, lower alkyl, substituted or unsubstituted aryl, or substituted or unsubstituted heteroaryl, provided that Rₐ and R_{b} are not both OH;

R₁-R₄ are independently for each occurrence H, halogen, lower alkyl, or haloalkyl;

R₅ is H, lower alkyl, or substituted or unsubstituted aryl, or substituted or unsubstituted heteroaryl;

m is 1-3; and

n is 0-3;

and pharmaceutically acceptable esters, salts, and prodrugs thereof.

In a further aspect, the invention provides compounds represented by Formula IV:

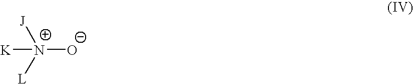

wherein
J, K, and L are independently for each occurrence alkyl, substituted alkyl, or —(CR₁R₂)ₙ-E-(CR₃R₄)ₘ—X;
X is H or CH₃;
R is independently for each occurrence H, lower alkyl, aryl, heterocyclyl;
R₁-R₄ are independently for each occurrence H, lower alkyl, fluoro or haloalkyl;
R₅ is independently for each occurrence H, lower alkyl, aryl, heterocyclyl;
m is independently for each occurrence 0-3;
n is independently for each occurrence 1-3;
E is O, S, SO, SO₂, —SO₂N(R₅)—, —N(R₅)SO₂—, NR₅, —C(O)O—, —O(O)C—, —CON(R₅)—, —N(R₅)CO—, or is absent;
   provided that when m=0, E is selected from O, NR₅R₆, CONR₅R₆, SO₂NR₅R₆; and
   R₆=H, lower alkyl, aryl, heterocyclyl;
   and pharmaceutically acceptable esters, salts, and prodrugs thereof. In certain preferred embodiments, the compound of the invention is not trimethylamine oxide or a compound represented by the formula:

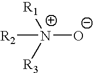

wherein

R₁, R₂, and R₃ are independently hydrogen, halogen, or lower C₁-C₆ alkyl;
or a pharmaceutically-acceptable salt thereof; or a mixture thereof. In certain preferred embodiments, the compound of the invention is not trimethylamine oxide or a compound represented by the formula:

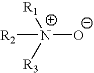

wherein

R₁, R₂, and R₃ are independently hydrogen, halogen, or lower C₁-C₆ alkyl;

or a pharmaceutically-acceptable salt thereof; or a mixture thereof.

In addition, the invention also pertains to pharmaceutical compositions. Such compositions comprise an effective amount of a compound of the invention, e.g. a compound of Formulae I-IV or otherwise described herein, and a pharmaceutically acceptable carrier.

In another embodiment, the invention provides a packaged formulation which includes a pharmaceutical composition comprising a compound of the invention, e.g., a compound of Formulae I-IV or otherwise described herein, and a pharmaceutically-acceptable carrier packaged with instructions for use in the treatment of a condition associated with ER stress, e.g., a condition selected from the group consisting of obesity, insulin resistance, hyperglycemia and type 2 diabetes. For example, the invention provides a packaged formulation which includes a pharmaceutical composition comprising a compound of the invention, e.g., a compound of Formulae I-IV or otherwise described herein, and a pharmaceutically-acceptable carrier packaged with instructions for use in the treatment of hypercholesterolemia, atherosclerosis, and related conditions.

ER Stress Related Disorders

The present invention further provides methods for treating or preventing a condition related to ER stress. In preferred embodiments, the condition is selected from the group consisting of obesity, insulin resistance, hyperglycemia and type 2 diabetes.

The method comprises administering to a subject, e.g., in need of such treatment or prevention, a compound of the invention, i.e., a compound of Formulae I-IV, or otherwise described herein.

For example, one aspect of the invention relates to the treatment or prevention of a condition related to ER-stress in a subject, e.g., in need thereof, comprising administering to said subject an effective amount of a compound represented by Formula II:

A-B-D (II)

wherein:

A is substituted or unsubstituted aryl, or substituted or unsubstituted heteroaryl;

B is alkyl, substituted alkyl, or —(CR₁R₂)ₙ-E-(CR₃R₄)ₘ—;

D is CO₂R, CONRₐR_{b}, SO₃R, SO₂NRₐR_{b}, tetrazolyl, —B(OR)(OR″), —P(O)R′OR″ or PO₃R′R″;

E is O, S, SO, SO₂, —SO₂N(R₅)—, —N(R₅)SO₂—, NR₅, —C(O)O—, —O(O)C—, —CONR₅—, —NR₅CO—, or is absent;

R, R′ and R″ are independently for each occurrence H or lower alkyl;

Rₐ and R_{b} are independently for each occurrence H, Off, lower alkyl, substituted or unsubstituted aryl, or substituted or unsubstituted heteroaryl, provided that Rₐ and R_{b} are not both OH;

R₁-R₄ are independently for each occurrence H, halogen, lower alkyl, or haloalkyl;

R₅ is H, lower alkyl, or substituted or unsubstituted aryl, or substituted or unsubstituted heteroaryl;

m is 1-3; and

n is 0-3;

and pharmaceutically acceptable esters, salts, and prodrugs thereof. In certain preferred embodiments of the invention, the compound of Formulas II-IIIb is not represented by the following formula:

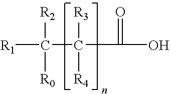

wherein

n is 1 or 2;

R₀ is aryl, heteroaryl, or phenoxy, the aryl and phenoxy being unsubstituted or substituted with, independently, one or more halogen, hydroxy or lower alkyl;

R₁ and R₂ are independently H, lower alkoxy, hydroxy, lower alkyl or halogen; and

R₃ and R₄ are independently H, lower alkyl, lower alkoxy, or halogen; or a pharmaceutically-acceptable derivative or salt thereof.

Additionally, another embodiment of the invention pertains to a method as described above, further comprising identifying a subject in need of prevention or treatment for ER stress-related diseases or conditions. In another embodiment, the invention provides a method as described above, further comprising the step of obtaining a compound of the invention. In one embodiment of the methods described herein, the subject is a mammal. In a further embodiment, the subject is a human.

Another embodiment of the invention provides a method for a method of screening for agents that reduce ER stress. The identified agents are useful in the treatment of obesity, type 2 diabetes, hyperglycemia, and insulin resistance. Agents to be screened are contacted with cells experiencing ER stress. The ER stress experienced by the cells may be caused by genetic alteration or treatment with a chemical compounds known to cause ER stress (e.g., tunicamycin, thapsigargin). Cells particularly useful in the inventive screen include liver cells and adipose cells. The levels of ER stress markers are then determined to identify agents that reduce ER stress. Examples of markers of ER stress include spliced forms of XBP-1, the phosphorylation status of PERK (Thr980) and eIF2a (Ser51), mRNA and protein levels of GRP78BIP, and JNK activity. Agents that when contacted with a cell with ER stress cause a reduction in the markers of ER stress as compared to an untreated control cell are identified as agents that reduce ER stress. A decrease in the levels of an ER stress marker are indicative of an agent that is useful in treating diseases associated with ER stress, such as obesity, type 2 diabetes, insulin resistance, hyperglycemia, cystic fibrosis, and Alzheimer's diseases. Agents identified using the inventive method are part of the invention. These agents may be further tested for use in pharmaceutical compositions.

In another aspect, the invention provides a method of diagnosing insulin resistance, hyperglycemia, or type 2 diabetes by measuring the level of expression of ER stress markers. Markers which may be analyzed in the inventive diagnostic method include spliced forms of XBP-1, phosphorylation status of PERK, phosphorylation of eIF2a, mRNA levels of GRP78BIP, protein levels of GRP78BIP, and JNK activity. Any other cellular marker known to be indicative of ER stress may also be used. The levels of these markers may be measured by any method known in the art including western blot, northern blot, immunoassay, or enzyme assay. An increase in the level of an ER stress markers indicates that the subject it at risk for insulin resistance, hyperglycemia, or type 2 diabetes.

Hypercholesterolemia, Atherosclerosis, and Related Conditions

The invention also provides methods for treating or preventing hypercholesterolemia, atherosclerosis, and related conditions. The method comprises administering to a subject, e.g., in need of such treatment or prevention, a compound of the invention, i.e., a compound of Formulae I-IV, or otherwise described herein. The methods of the invention can be used to treat or prevent atherosclerosis, stroke, and other ischemic vascular diseases, dyslipidemia and hypercholesterolemia, and prevent complications of these conditions.

For example, the invention pertains to treatment or prevention of hypercholesterolemia, atherosclerosis, and related conditions by administering compounds represented by Formula I:

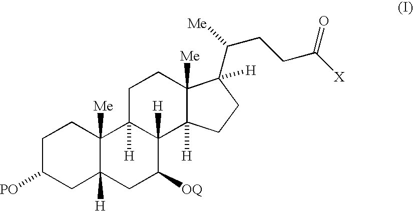

in which
P and Q are independently for each occurrence H, lower alkyl, PO₃R₂, SO₃R or COR;
X is OR, NR₂ or NR—B-D;
B is alkylene, substituted alkylene, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″, or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R is H, lower alkyl, aryl, or heterocyclyl;
R₁-R₄ are independently for each occurrence H, lower alkyl, fluoro or haloalkyl;
R₅ is H, lower alkyl, or substituted or unsubstituted aryl, or substituted or unsubstituted heteroaryl;
m is 1-3; and
n is 0-3;
   and pharmaceutically acceptable esters, salts, and prodrugs thereof. In certain preferred embodiments, the compound of the invention is not tauroursodeoxycholic acid (TUDCA) or a TUDCA analog represented by the formula:

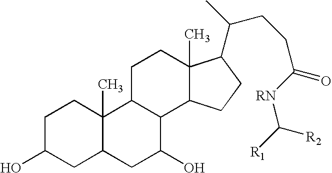

in which:

R is —H or C₁-C₄ alkyl;

R₁ is CH₂—SO₃R₃ and R₂ is —H; or R₁ is —COOH and R₂ is CH₂—CH₂—CONH₂, —CH₂—CONH₂, —CH₂—CH₂—SCH₃ or CH₂—S—CH₂—COOH; and

R₃ is —H or a basic amino acid; or a pharmaceutically acceptable salt thereof.

In one embodiment, the invention provides a method as described above, further comprising identifying a subject in need of prevention or treatment for hypercholesterolemia, atherosclerosis, and related conditions. In another embodiment, the invention provides a method as described above, further comprising the step of obtaining a compound of the invention. In one embodiment of the methods described herein, the subject is a mammal. In a further embodiment, the subject is a human.

Moreover, it should be recognized that, in certain embodiments, the methods of treatment or prevention of ER stress related disorders or conditions, such as hypercholesterolemia, atherosclerosis, and related conditions, is not dependent upon the mechanism of treatment or prevention, but rather resulting relief or prevention of one or more symptoms of the disorder or condition.

### DETAILED DESCRIPTION OF THE INVENTION

### 1. Definitions

Before further description of the present invention, and in order that the invention may be more readily understood, certain terms are first defined and collected here for convenience.

The term “administration” or ‘administering’ includes routes of introducing the compound(s) to a subject to perform their intended function. Examples of routes of administration which can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound can be coated with or disposed in a selected material to protect it from natural conditions which may detrimentally effect its ability to perform its intended function. The compound can be administered alone, or in conjunction with either another agent as described above or with a pharmaceutically-acceptable carrier, or both. The compound can be administered prior to the administration of the other agent, simultaneously with the agent, or after the administration of the agent. Furthermore, the compound can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms re placing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g. oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “biological activities” includes all activities elicited by compounds in a responsive cell. It includes genomic and non-genomic activities elicited by these compounds (Gniadecki R. and Calverley M. J. (1998) Pharmacology & Toxicology 82: 173-176; Bouillon, R. et al. (1995) Endocrinology Reviews 16(2):206-207; Norman A. W. et al. (1992) J. Steroid Biochem Mol. Biol. 41:231-240; Baran D. T. et al. (1991) J. Bone Miner Res. 6:1269-1275; Caffrey J. M. and Farach-Carson M. C. (1989) J. Biol. Chem. 264:20265-20274; Nemere I. et al. (1984) Endocrinology 115:1476-1483).

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “effective amount” includes an amount effective, at dosages and for periods of time necessary, to achieve the desired result (e.g., sufficient to treat a state associated with ER stress in a cell or sufficient to treat hypercholesterolemia, atherosclerosis, and related conditions). An effective amount of a compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the compound are outweighed by the therapeutically beneficial effects.

A therapeutically effective amount of compound (i.e., an effective dosage) will typically be from 5 mg/kg/day to 5 g/kg/day, more preferably 5 mg/kg/day to 1 g/kg/day, and still more preferably 10 mg/kg/day to 500 mg/kg/day. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. It will also be appreciated that the effective dosage of a compound used for treatment may increase or decrease over the course of a particular treatment.

The language “a disorder related to ER stress,” “a condition related to ER stress,” “an ER stress related disorder,” “ER-stress associated state,” and “ER-stress state” are described and used interchangeably herein.

The term “enantiomers” refers to two stereoisomers of a compound which are non-superimposable mirror images of one another. An equimolar mixture of two enantiomers is called a “racemic mixture” or a “racemate.”

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl. The term “hydroxyl” means —OH.

The term “heteroatom” as used herein means an atom of any element other than carbon or hydrogen. Preferred heteroatoms are nitrogen, oxygen, sulfur and phosphorus.

The term “hypercholesterolemia” refers to the presence of high or excessive levels of cholesterol in the blood. In particular embodiments, “hypercholesterolemia” refers to fasting total cholesterol levels above 200 mg/dL.

The language “improved biological properties” refers to any activity inherent in a compound of the invention that enhances its effectiveness in vivo. En a particular embodiment, this term refers to any qualitative or quantitative improved therapeutic property of a compound, such as reduced toxicity.

The term “isomers” or “stereoisomers” refers to compounds which have identical chemical constitution, but differ with regard to the arrangement of the atoms or groups in space.

The term “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g., a therapeutic result.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g. acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “reduced toxicity” is intended to include a reduction in any undesired side effect elicited by a compound when administered in vivo.

The term “sulfhydryl” or “thiol” means —SH.

The term “subject” includes organisms which are capable of an endoplasmic reticulum cellular stress response (e.g., suffering from an ER-stress associated state, i.e., suffering from hypercholesterolemia, atherosclerosis, and related conditions) or who could otherwise benefit from the administration of a compound of the invention, such as human and non-human animals. Preferred human animals include human patients suffering from or prone to suffering from an ER-stress associated state, i.e., hypercholesterolemia, atherosclerosis, and related conditions, as described herein. The term “non-human animals” of the invention includes all vertebrates, e.g., mammals, e.g., rodents, e.g., mice, and non-mammals, such as non-human primates, sheep, dog, cow, chickens, amphibians, reptiles, etc.

As used herein, the term “obtaining” includes purchasing, synthesizing, isolating or otherwise acquiring one or more of the compounds used in practicing the invention.

The phrases “systemic administration,” “administered systemically”, “peripheral administration” and “administered peripherally” as used herein mean the administration of a compound(s), drug or other material, such that it enters the patient's system and, thus, is subject to metabolism and other like processes, for example, subcutaneous administration.

As used herein, the term “pharmaceutically acceptable salt,” is a salt formed from an acid and a basic group of one of the disclosed compounds. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, furmarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt prepared from a disclosed compound having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

As used herein, the term “pharmaceutically acceptable ester,” refers to esters including, but not limited to, lower alkyl esters such as methyl, ethyl, isopropyl, and the like.

With respect to the nomenclature of a chiral center, terms “d” and “1” configuration are as defined by the IUPAC Recommendations. As to the use of the terms, diastereomer, racemate, epimer and enantiomer will be used in their normal context to describe the stereochemistry of preparations.

### 2. Compounds

Compounds of the invention include tauroursodeoxycholic acid (TUDCA) related analogs, 4-phenyl butyrate (PBA) related analogs, and tertiary amine N-oxides (TMAOs). Such compounds, as noted herein, have been identified herein for their use in treating or preventing conditions or disorders related to ER stress, i.e., hypercholesterolemia, atherosclerosis, and related conditions.

A. Analogs of TUDCA

In one embodiment, the invention provides compounds represented by Formula I:

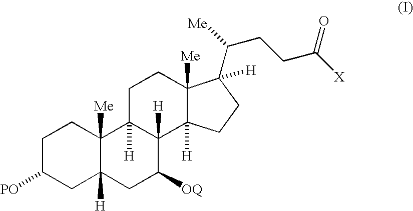

in which

P and Q are independently for each occurrence H, lower alkyl, PO₃R₂, SO₃R or COR;
X is OR, NRₐR_{b} or NR—B-D;
B is alkylene, substituted alkylene, or —(CR₁R₂)ₙ-E-(CR₃R₄)ₘ—);
D is CO₂R, CONRₐR_{b}, SO₃R, SO₂NRₐR_{b}, tetrazolyl, —B(OR)(OR″), —P(O)R′OR″, or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R is H, lower alkyl, aryl, or heterocyclyl;
R₁-R₄ are independently for each occurrence H, lower alkyl, fluoro or haloalkyl;
R₅ is H, lower alkyl, or substituted or unsubstituted aryl, or substituted or unsubstituted heteroaryl;
m is 1-3; and
n is 0-3;
   and pharmaceutically acceptable esters, salts, and prodrugs thereof. In certain preferred embodiments, the compound of the invention is not tauroursodeoxycholic acid (TUDCA) or a TUDCA analog represented by the formula:

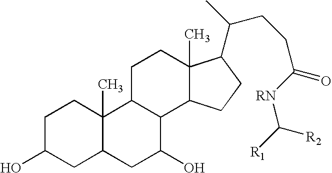

in which:

R is —H or C₁-C₄ alkyl;

R₁ is CH₂—SO₃R₃ and R₂ is —H; or R₁ is COOH and R₂ is CH₂—CH₂—CONH₂, —CH₂—CONH₂, —CH₂—CH₂—SCH₃ or CH₂—S—CH₂—COOH; and

R₃ is —H or a basic amino acid; or a pharmaceutically acceptable salt thereof.

In certain embodiments directed to novel compounds of the present invention, the compound is not one of the following compounds:

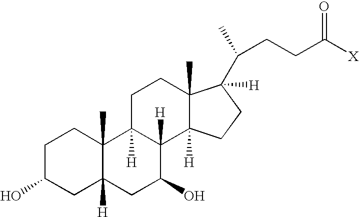

in which X is one of the following moieties:

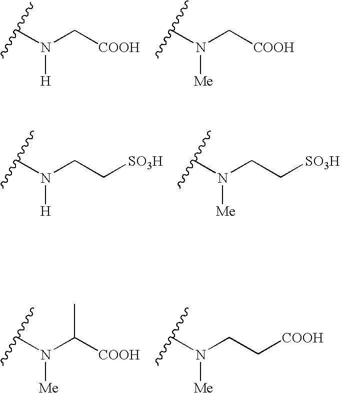

In certain embodiments of the invention directed to the methods of treatment or prevention, the compound may be one of the following compounds:

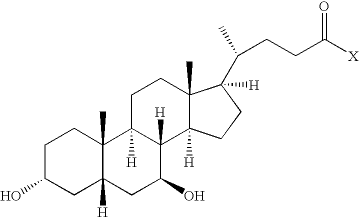

in which X is one of the following moieties:

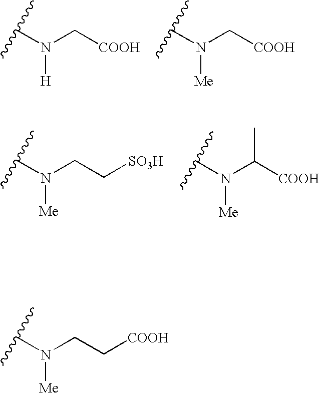

Various preferred embodiments of this aspect of the invention include individual compounds of Formula I wherein: P and Q are each H; X is NR—B-D; B is alkylene or substituted alkylene; D is CO₂R, CONRₐR_{b}, or PO₃RₐR_{b}; R is H or lower alkyl.

In particular embodiments, the compounds of the invention may be selected from the exemplary compound listing shown below. Importantly, it should be noted that the tabular listing below is used merely as a convenience, and each compound below should be considered a separate embodiment of the invention:

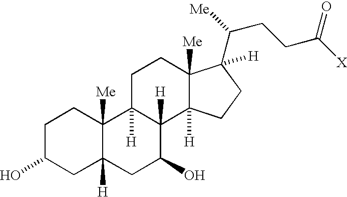

where X is:

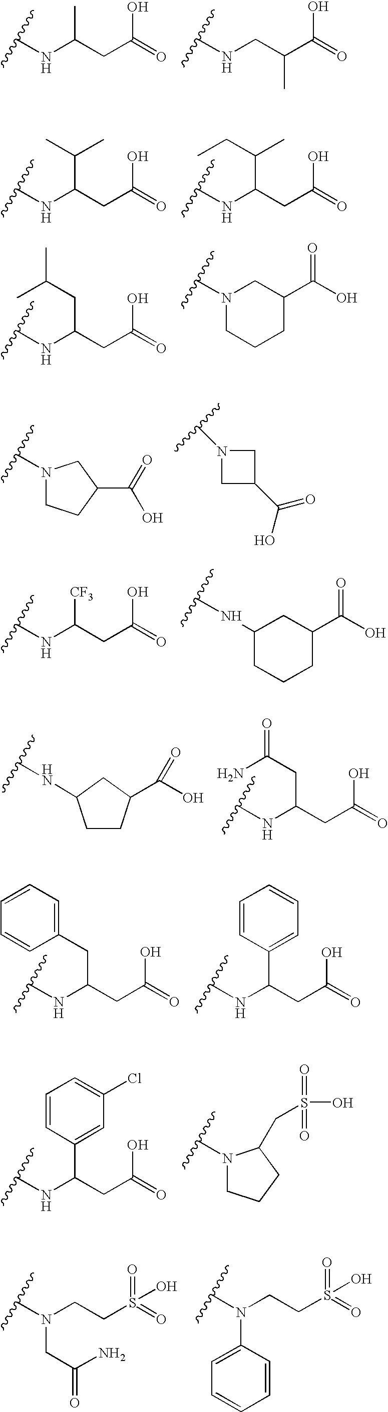

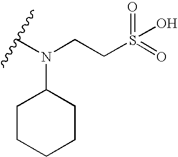

In another embodiment, the compound of the invention may be represented by the following formula

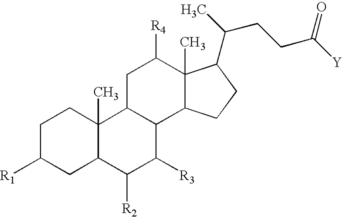

wherein
R₁ is selected from the group consisting of H, —OH, and ═O;
R₂ is selected from the group consisting of H, and OH;
R₃ is selected from the group consisting of H, —OH, and ═O;
R₄ is selected from the group consisting of H, —OH, and ═O;
Y is selected from the group consisting of OR₅, and NR₆R₇;
R₅ is selected from the group consisting of H, and —CH₃;
R₆ is selected from the group consisting of —CH₂C(O)OH, —CH₂CH₂S(O)₂OH, and —C(R₈)H—C(R₉)H—C(O)OR₁₀;
R₇ is selected from the group consisting of H, isopropyl, isobutyl, methyl, ethyl, phenyl, cyclohexyl, benzyl, methoxy-ethyl, and acetamide, or R₉, R₇ and the nitrogen to which it is attached form a heterocyclic ring comprising about 4 to 6 atoms;
R₈ is selected from the group consisting of H, —CH₃, and isopropyl;
R₉ is selected from the group consisting of H, and —CH₃, or R₉, R₇ and the nitrogen to which it is attached form a heterocyclic ring comprising about 4 to 6 atoms;
R₁₀ is selected from the group consisting of H, —CH₃, and —CH₂CH₃.

Additional particular embodiments are shown in the table below. Again, it should be noted that the tabular listing below is used merely as a convenience, and each compound below should be considered a separate embodiment of the invention:

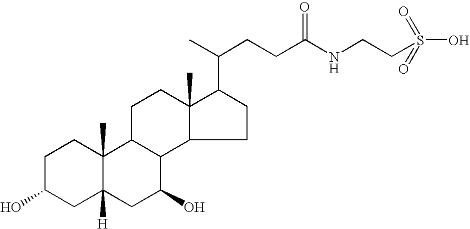

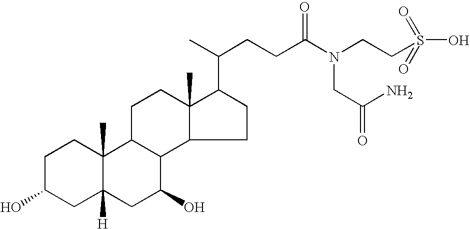

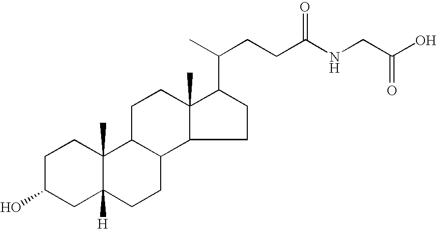

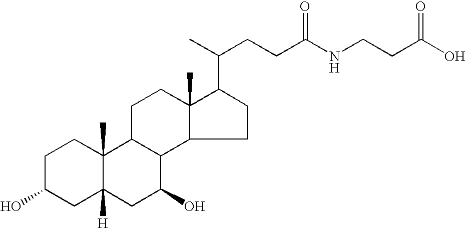

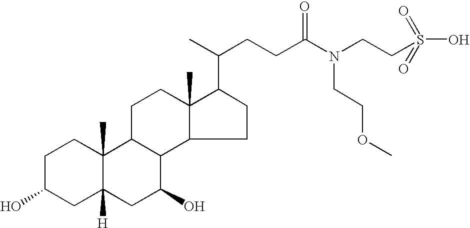

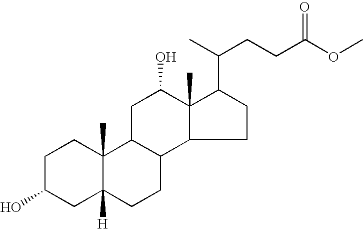

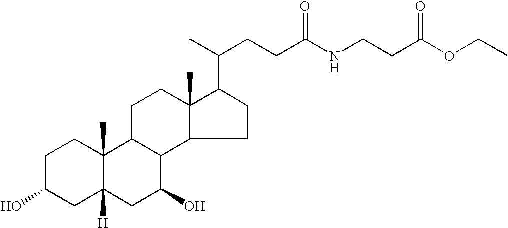

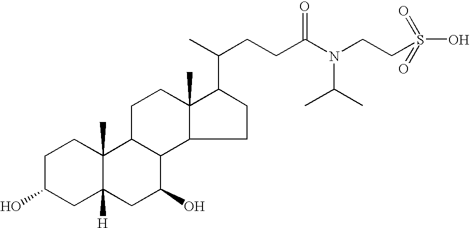

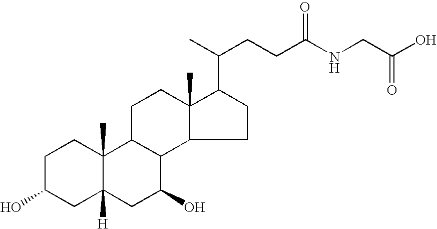

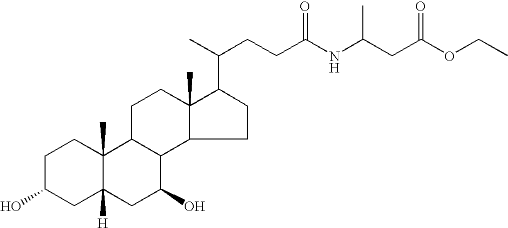

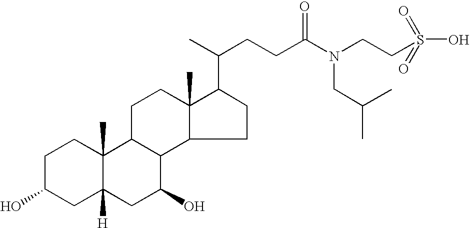

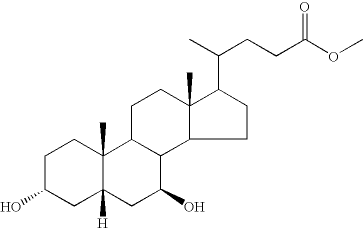

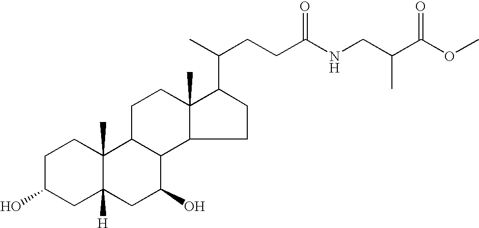

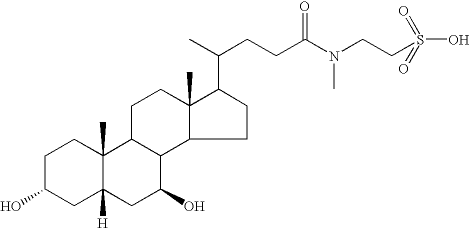

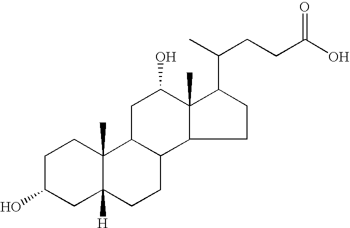

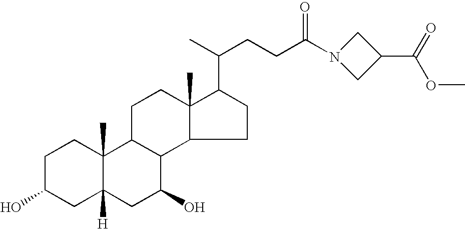

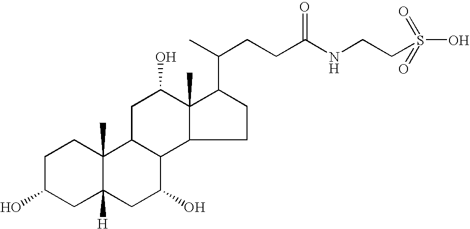

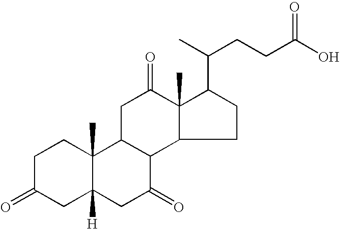

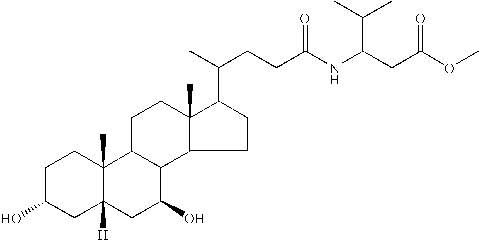

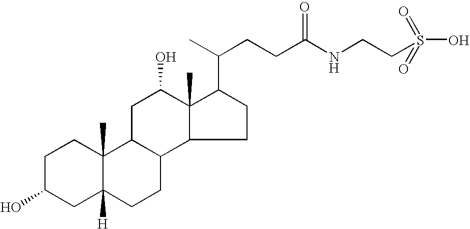

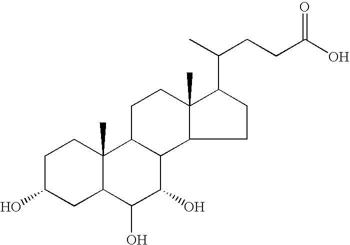

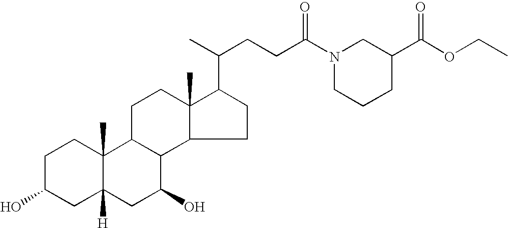

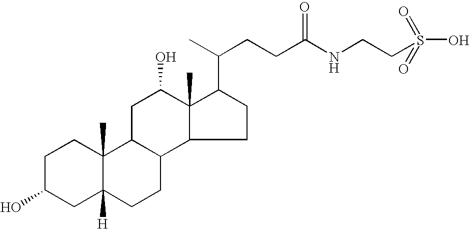

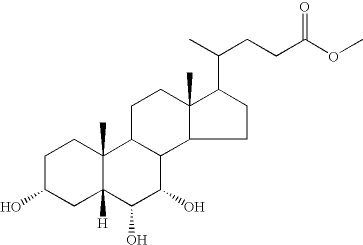

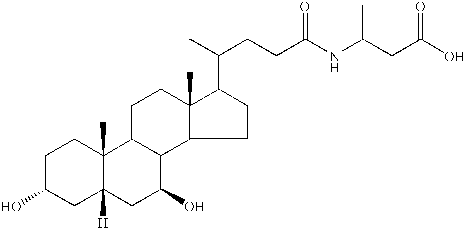

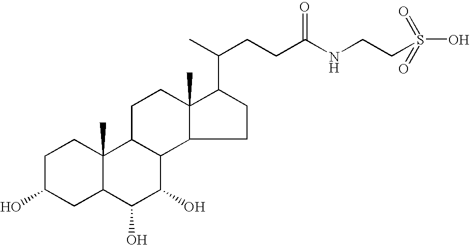

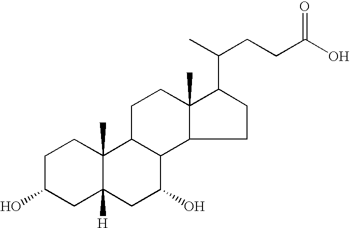

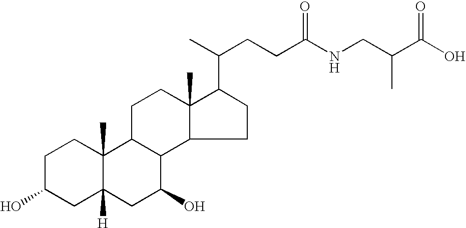

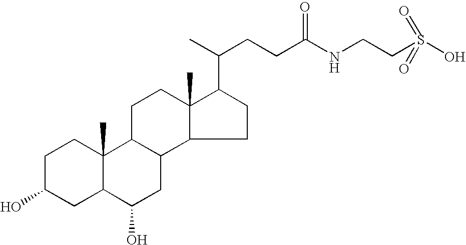

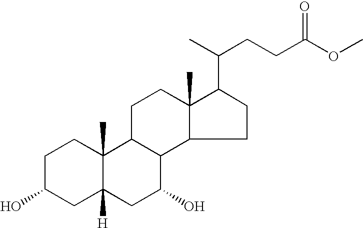

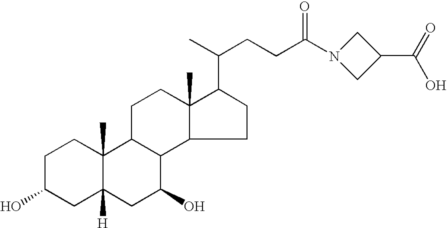

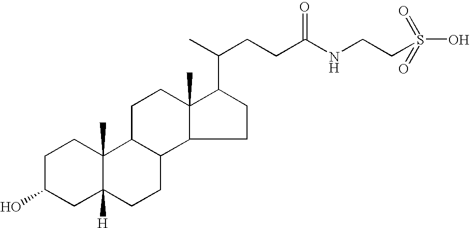

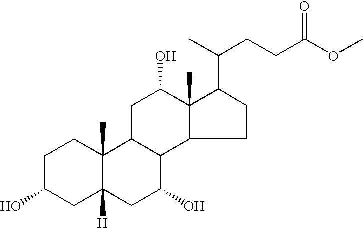

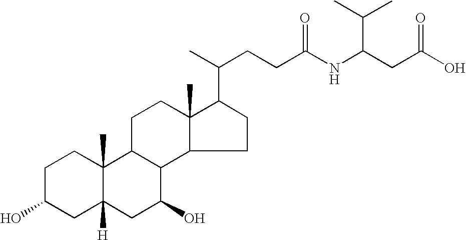

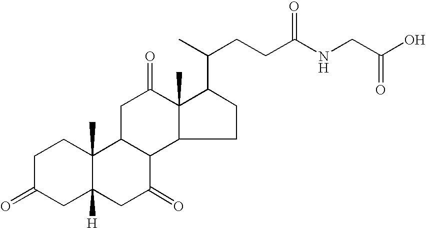

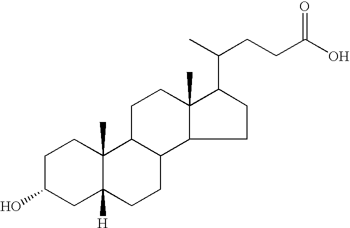

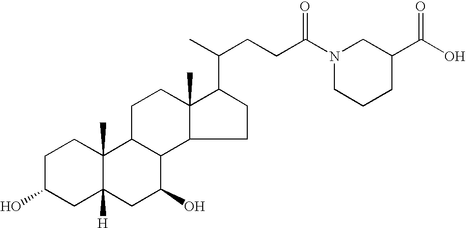

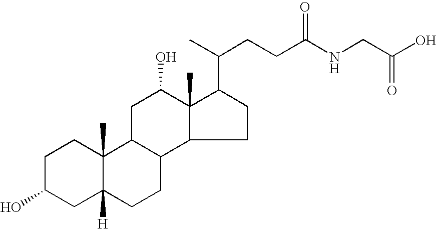

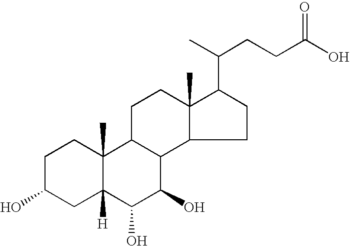

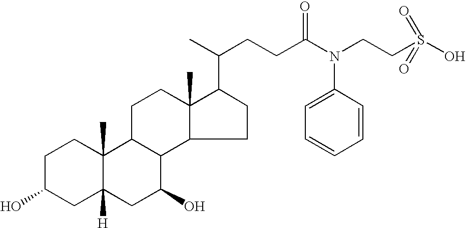

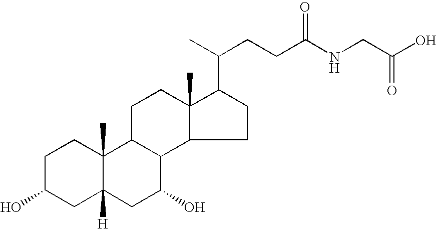

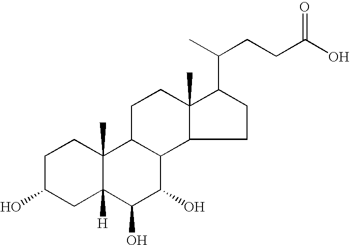

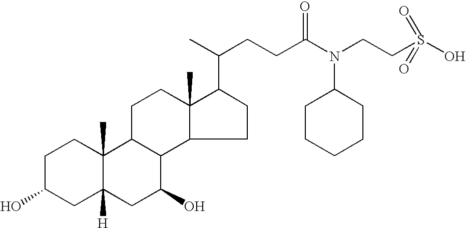

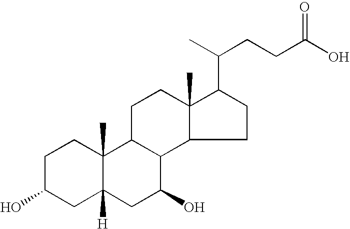

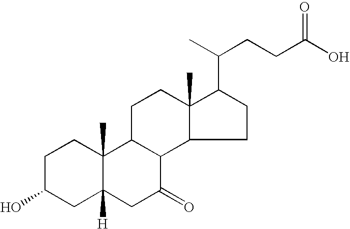

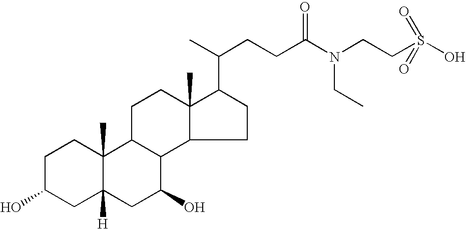

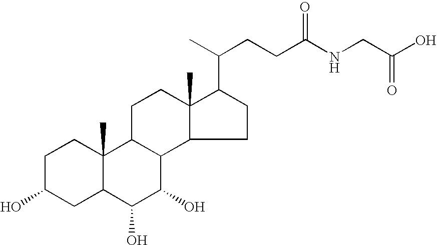

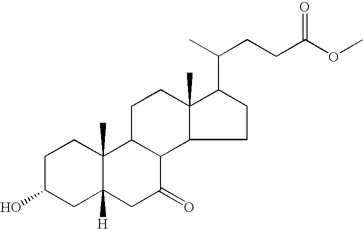

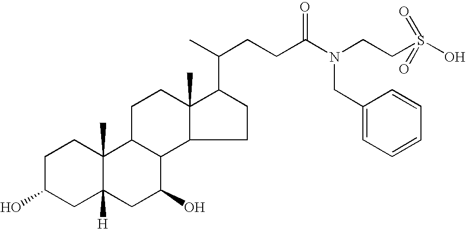

Further particular embodiments are shown below:

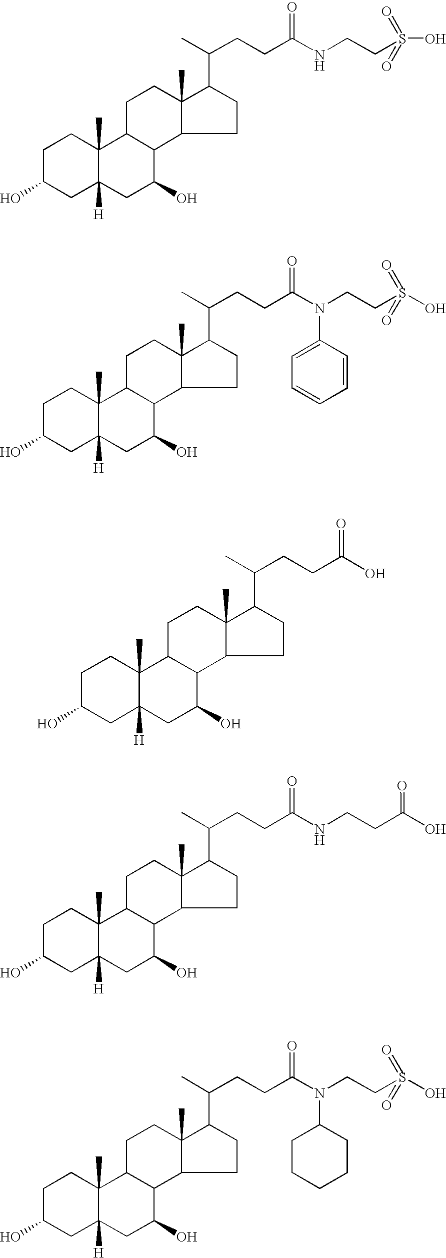

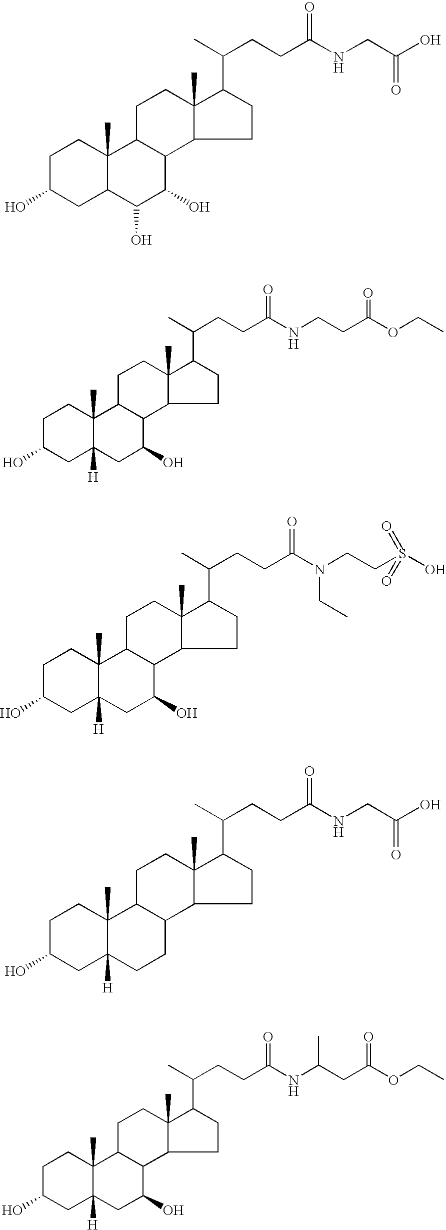

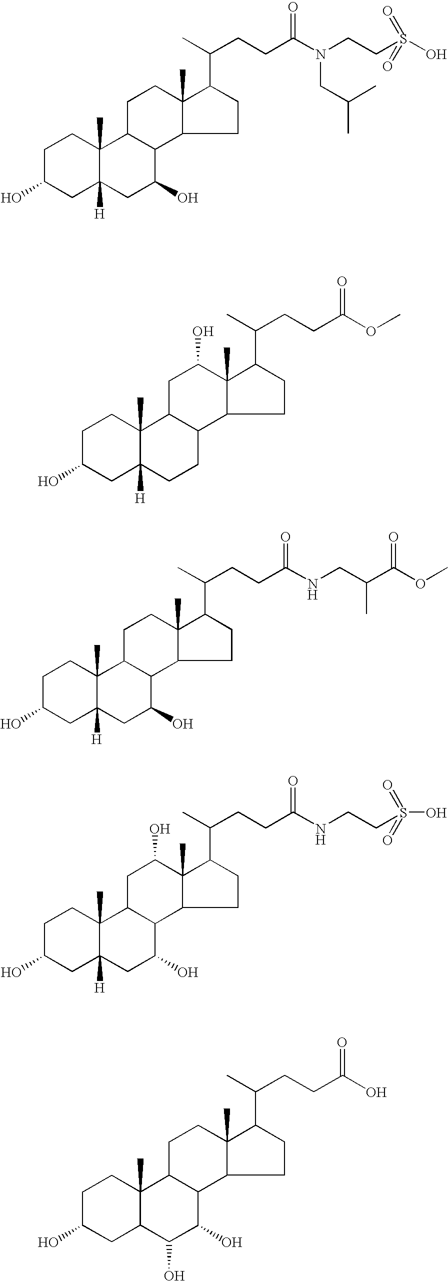

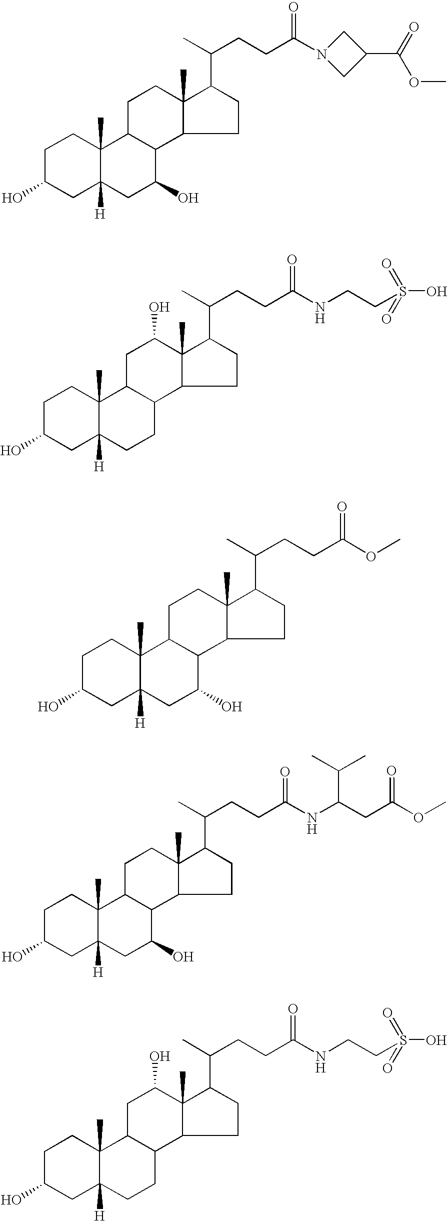

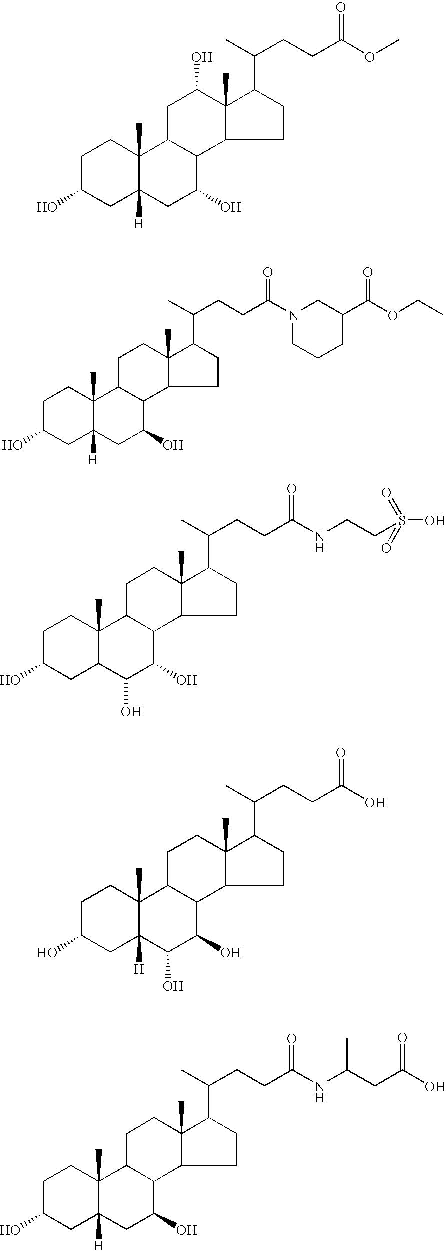

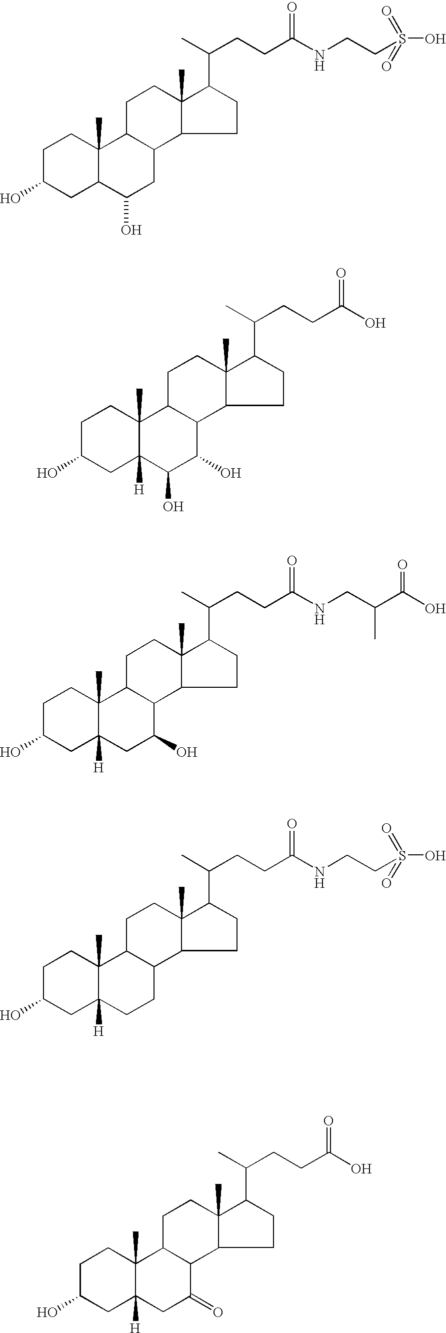

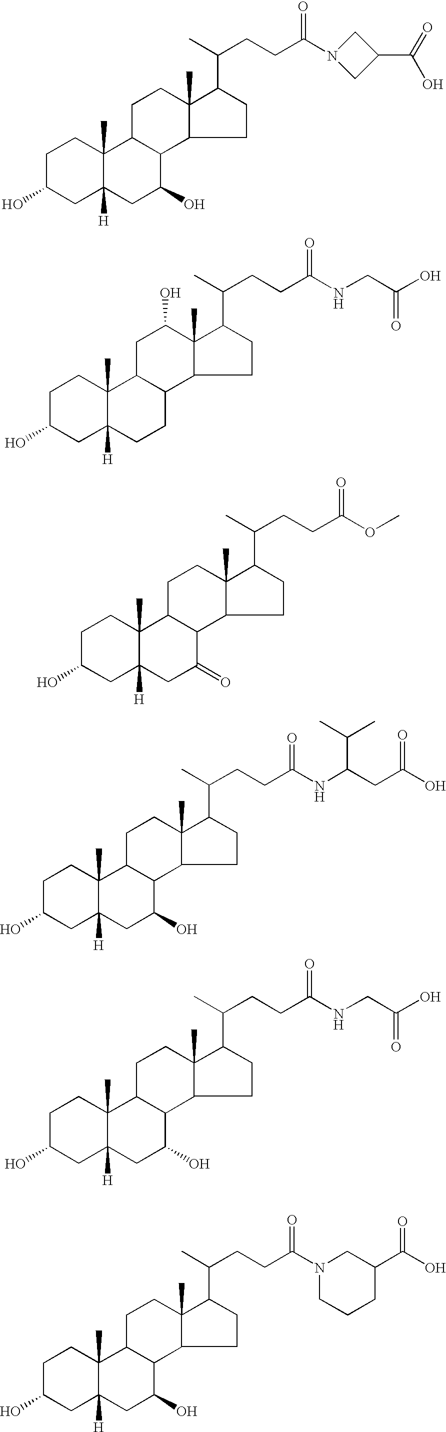

In certain embodiments, the compound may be one of the following compounds

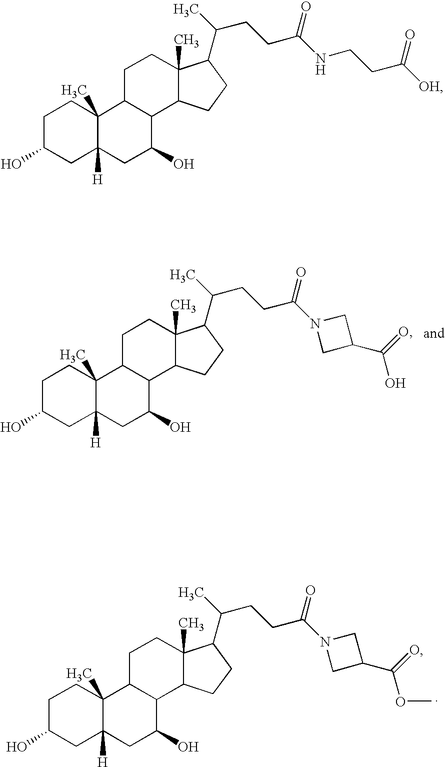

In certain embodiments of the invention, the compound of the invention is not a compound selected from the group consisting of

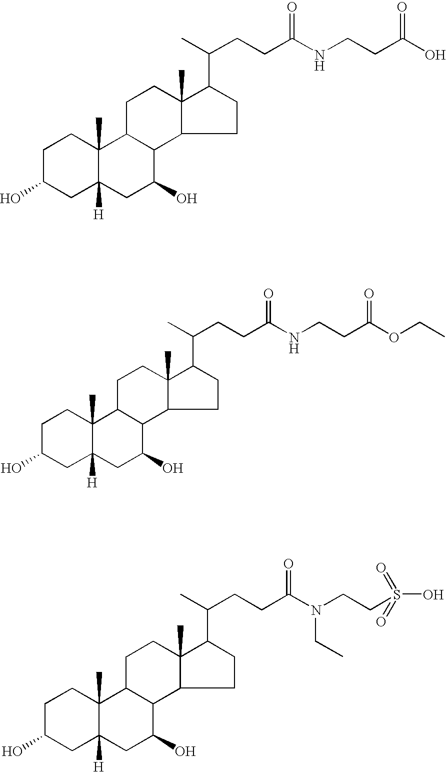

B. Analogs of PBA

In another embodiment, the invention provides compounds represented by Formula EC:

A-B-D (II)

wherein:

A is substituted or unsubstituted aryl, or substituted or unsubstituted heteroaryl;

B is alkyl, substituted alkyl, or —(CR₁R₂)ₙ-E-(CR₃R₄)ₘ—;

D is CO₂R, CONRₐR_{b}, SO₃R, SO₂NRₐR_{b}, tetrazolyl, —B(OR)(OR″), —P(O)R′OR″ or PO₃R′R″;

E is O, S, SO, SO₂, —SO₂N(R₅)—, —N(R₅)SO₂—, NR₅, —C(O)O—, —O(O)C—, —CONR₅—, —NR₅CO—, or is absent;

R, R′ and R″ are independently for each occurrence H or lower alkyl;

Rₐ and R_{b} are independently for each occurrence H, OH, lower alkyl, substituted or unsubstituted aryl, or substituted or unsubstituted heteroaryl, provided that & and R_{b} are not both OH;

R₁-R₄ are independently for each occurrence H, halogen, lower alkyl, or haloalkyl;

R₅ is H, lower alkyl, or substituted or unsubstituted aryl, or substituted or unsubstituted heteroaryl;

m is 1-3; and

n is 0-3;

and pharmaceutically acceptable esters, salts, and prodrugs thereof. In preferred embodiments, the compound of Formulas II-IIIb is not 4-phenylbutyric acid (PBA). In certain preferred embodiments, the compound of Formulas II-IIIb is not represented by the following formula:

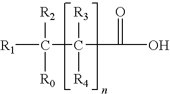

wherein

n is 1 or 2;

R₀ is aryl, heteroaryl, or phenoxy, the aryl and phenoxy being unsubstituted or substituted with, independently, one or more halogen, hydroxy or lower alkyl;

R₁ and R₂ are independently H, lower alkoxy, hydroxy, lower alkyl or halogen; and

R₃ and R₄ are independently H, lower alkyl, lower alkoxy, or halogen; or a pharmaceutically-acceptable derivative or salt thereof. In certain embodiments, A is substituted or unsubstituted aryl. In certain embodiments, B is —(CR₁R₂)ₙ-E-(CR₃R₄)ₘ, and in particular embodiments, at least one of R₁-R₄ may also be halogen, e.g., wherein at least one halogen is fluoro. In certain other embodiments, D is CONRₐR_{b}, SO₃R, SO₂NRₐR_{b}, tetrazolyl, —B(OR)(OR″), —P(O)R′OR″ or PO₃R′R″, e.g., D is SO₃R, wherein R is H. In yet other embodiments, E is O, S, —SO₂N(R₅)—, —N(R₅)SO₂—, NR₅, —C(O)O—, —O(O)C—, —CONR₅—, or —NR₅CO—, e.g., E is —CONR₅— or —NR₅CO—.

In another embodiment, the invention provides compounds of Formula II, represented by Formula IIa or IIb:

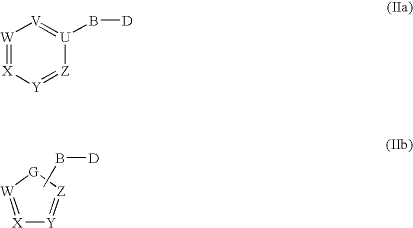

wherein:

U is C or N;

V, W, X, Y, and Z are independently for each occurrence CR₆, NR₇, O, or S.

G is NR₇, O, or S;

R₆ is independently for each occurrence H, halogen, alkyl, aryl, or heterocyclyl;

R₇ is H, alkyl, aryl, or heterocyclyl, or is absent;

B is alkyl, substituted alkyl, or —(CR₁R₂)ₙ-E-(CR₃R₄)ₘ—;

D is CO₂R, CONRₐR_{b}, SO₃R, SO₂NRₐR_{b}, tetrazolyl, —B(OR)(OR″), —P(O)R′OR″ or PO₃R′R″;

E is O, S, SO, SO₂, —SO₂N(R₅)—, —N(R₅)SO₂—, NR₅, —C(O)O—, —O(O)C—, —CONR₅—, —NR₅CO—, or is absent;

R, R′ and R″ are independently for each occurrence H or lower alkyl;

Rₐ and R_{b} are independently for each occurrence H, OH, lower alkyl, substituted or unsubstituted aryl, or substituted or unsubstituted heteroaryl, provided that Rₐ and R_{b} are not both OH;

R₁-R₄ are independently for each occurrence H, halogen, lower alkyl, or haloalkyl;

R₅ is H, lower alkyl, or substituted or unsubstituted aryl, or substituted or unsubstituted heteroaryl;

m is 1-3; and

n is 0-3;

and pharmaceutically acceptable esters, salts, and prodrugs thereof.

In another embodiment, the invention provides compounds of Formula II represented by Formula IIIa or IIIb:

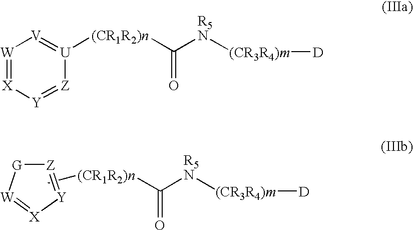

wherein

U is C or N;

V, W, X, Y, and Z are independently for each occurrence CR₆, NR₇, O, or S;

G is NR₇, O, or S;

R₆ is independently for each occurrence H, halogen, alkyl, aryl, or heterocyclyl;

R₇ is H, alkyl, aryl, or heterocyclyl, or is absent;

D is CO₂R, CONRₐR_{b}, SO₃R, SO₂NRₐR_{b}, tetrazolyl, —B(OR)(OR″), —P(O)R′OR″ or PO₃R′R″;

R, R′ and R″ are independently for each occurrence H or lower alkyl;

Rₐ and R_{b} are independently for each occurrence H, OH, lower alkyl, substituted or unsubstituted aryl, or substituted or unsubstituted heteroaryl, provided that Rₐ and R_{b} are not both OH;

R₁-R₄ are independently for each occurrence H, halogen, lower alkyl, or haloalkyl;

R₅ is H, lower alkyl, or substituted or unsubstituted aryl, or substituted or unsubstituted heteroaryl;

m is 1-3; and

n is 0-3;

and pharmaceutically acceptable esters, salts, and prodrugs thereof. In certain embodiments, at least one of R₁-R₄ is halogen, e.g., at least one halogen is fluoro.

Various preferred embodiments of this aspect of the invention include compounds of Formulas II-IIIb characterized by one or more of the following: U is C; at least two of W, X, Y and Z are CR₆; D is CONRₐR_{b}, SO₃R, SO₂NRₐR_{b}, tetrazolyl, —B(OR)(OR″), —P(O)R′OR″ or —PO₃R′R″, more preferably CONRₐR_{b}, SO₃R or —PO₃R′R″, still more preferably SO₃R, wherein R is H; E is O, S, —SO₂N(R₅)—, —N(R₅)SO₂—, NR₅, —C(O)O—, —O(O)C—, —CONR₅—, or —NR₅CO—, more preferably —CONR₅—, or —NR₅CO—. In certain preferred embodiments, at least one, and preferably two, of R₁-R₄ are halogen, more preferably fluoro.

In certain embodiments of the invention, the compounds of Formulas II-IIIb may be characterized by one or more of the following: U is C; at least two of W, X, Y and Z are CR₆; D is CONRₐR_{b}, SO₃R, SO₂NRₐR_{b}, tetrazolyl, —B(OR)(OR″), —P(O)R′OR″ or —PO₃R′R″, e.g., CONRₐR_{b}, SO₃R or —PO₃R′R″, e.g., SO₃R, wherein R is H; E is O, S, —SO₂N(R₅)—, —N(R₅)SO₂—, NR₅, —C(O)O—, —O(O)C—, —CONR₅—, or —NR₅CO—, e.g., —CONR₅—, or —NR₅CO—.

Selected, non-limiting compounds of the invention are shown in the following table. Importantly, it should be noted that the tabular listing below is used merely as a convenience, and each compound below should be considered a separate embodiment of the invention:

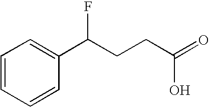

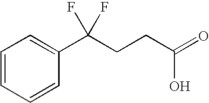

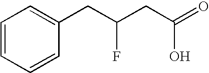

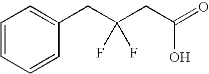

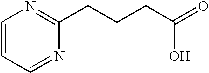

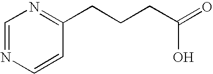

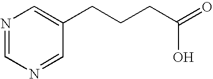

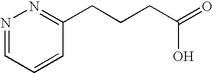

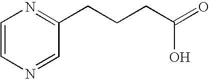

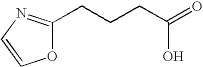

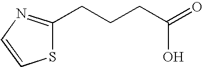

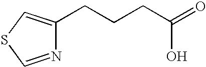

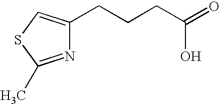

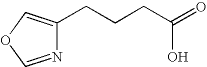

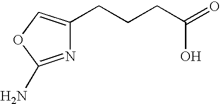

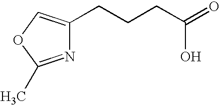

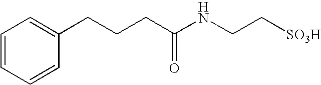

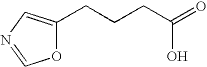

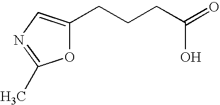

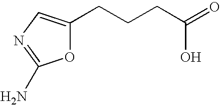

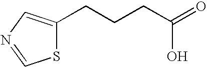

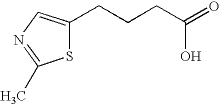

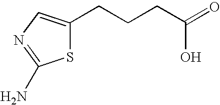

Selected, non-limiting compounds of the invention are shown in the following table. Importantly, it should be noted that the tabular listing below is used merely as a convenience, and each compound below should be considered a separate embodiment of the invention:

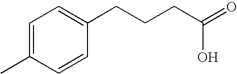

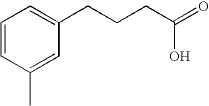

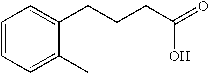

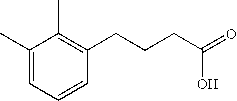

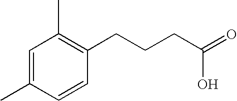

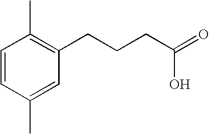

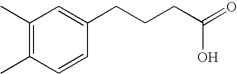

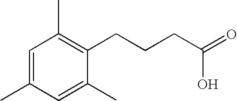

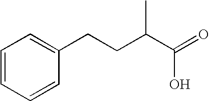

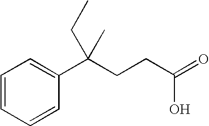

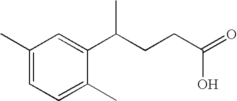

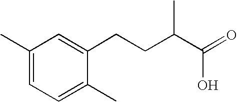

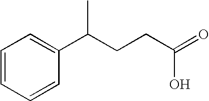

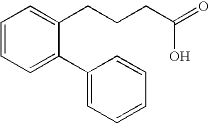

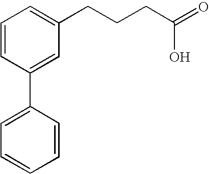

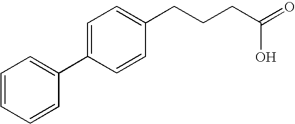

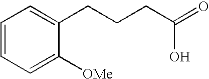

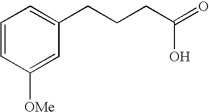

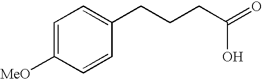

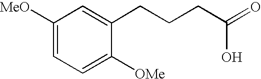

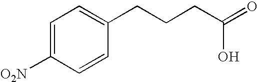

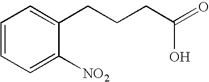

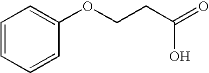

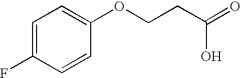

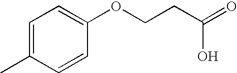

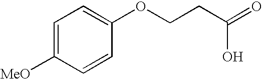

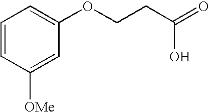

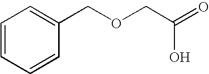

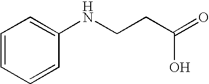

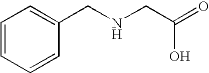

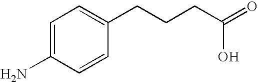

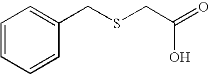

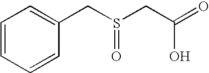

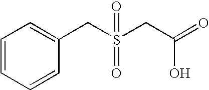

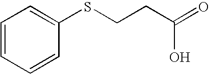

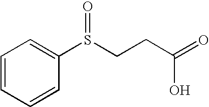

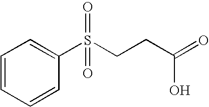

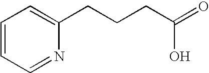

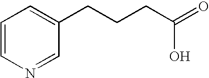

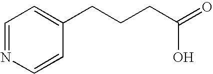

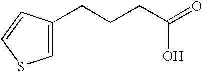

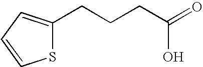

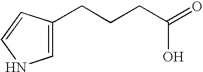

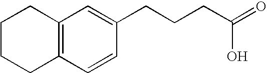

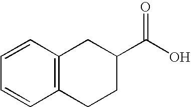

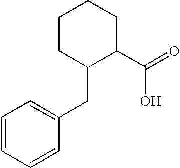

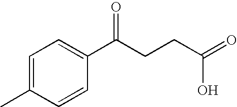

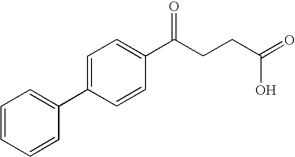

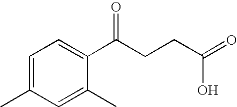

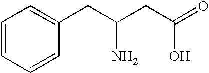

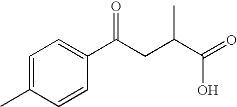

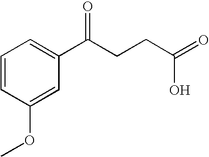

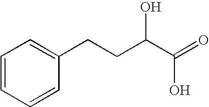

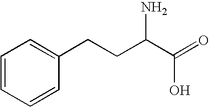

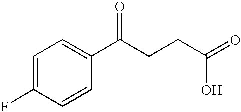

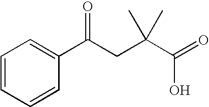

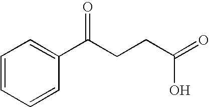

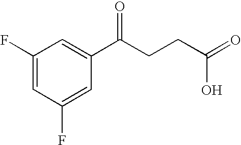

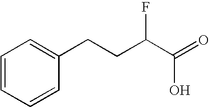

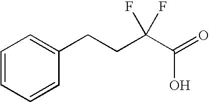

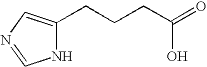

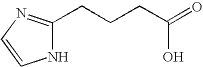

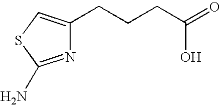

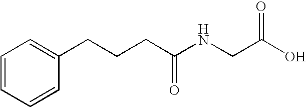

In another embodiment, the compound of the invention may be represented by the following formula

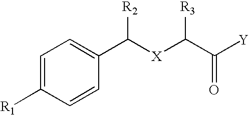

wherein
R₁ is selected from the group consisting of H, phenyl optionally substituted with halogen, and benzyl;
R₂ is selected from the group consisting of H and ═O;
R₃ is selected from the group consisting of H, halogen, OH, and ═O;
X is selected from the group consisting of —CH₂—, —CHF—, —O—, —C(O)—, and —C(OH)H—;
Y is selected from the group consisting of OR₄, and NR₅;
R₄ is selected from the group consisting of H, and lower alkyl;
R₅ is selected from the group consisting of —CH₂C(O)OR₆, is selected from the group consisting of —CH₂CH₂S(O)₂OH;
R₆ is selected from the group consisting of H, and lower alkyl

Additional particular embodiments are shown in the table below. Again, it should be noted that the tabular listing below is used merely as a convenience, and each compound below should be considered a separate embodiment of the invention:

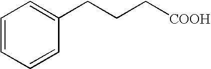

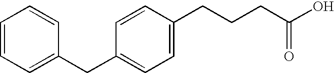

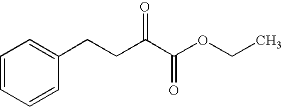

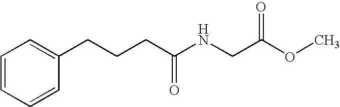

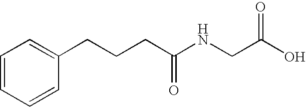

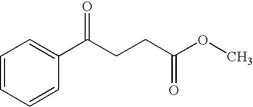

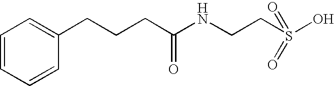

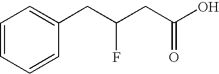

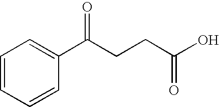

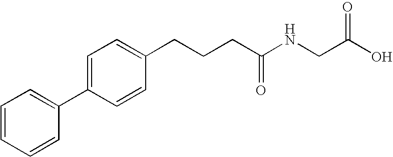

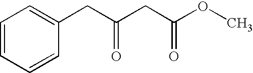

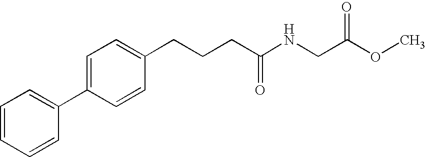

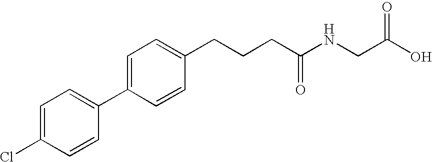

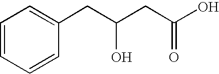

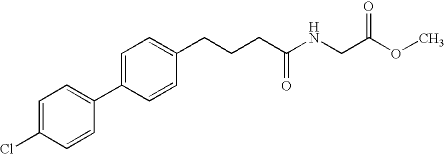

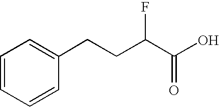

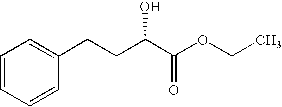

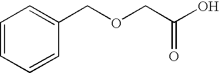

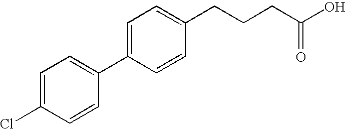

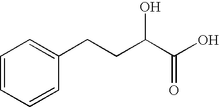

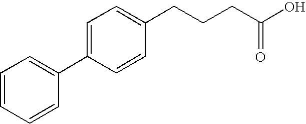

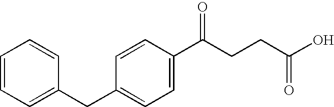

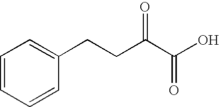

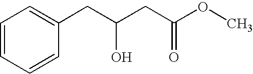

Additional particular embodiments are shown in the table below. Again, it should be noted that the tabular listing below is used merely as a convenience, and each compound below should be considered a separate embodiment of the invention:

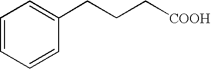

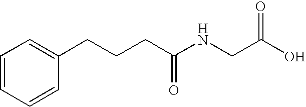

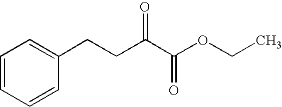

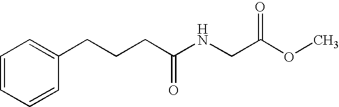

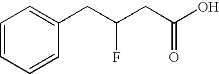

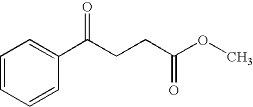

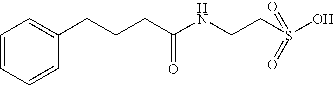

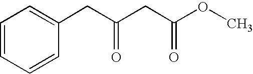

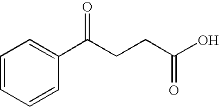

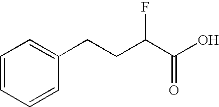

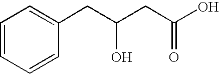

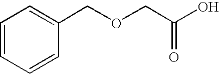

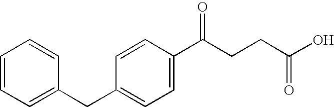

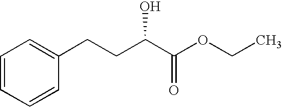

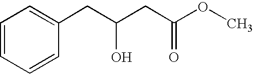

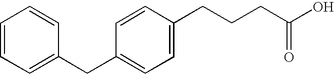

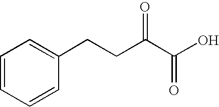

In certain embodiments, the compound may be one of the following compounds

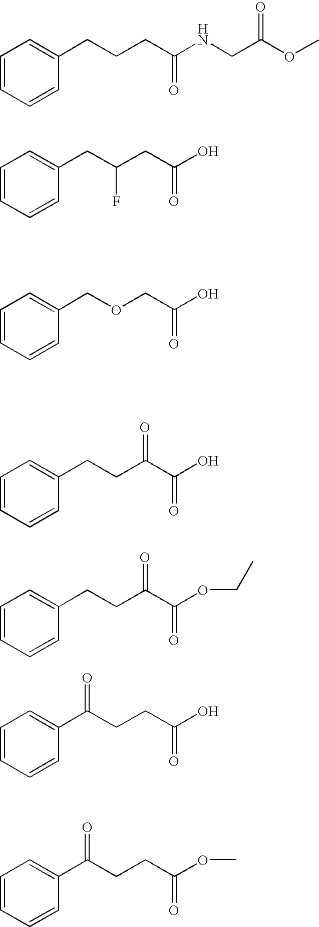

In yet another embodiment, the compound may be represented by the formula

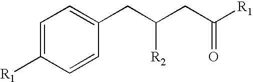

wherein
R₁ is selected from the group consisting of OH, —HNCH₂CH₂SO₃H, —HNCH₂CO₂H
R₂ is selected from the group consisting of H and F;
R₃ is selected from group consisting of H or 4-halophenyl
   provided that at least one of the R substituents is not H; and
   provided that when R₁ is OH, R₂ is F or R₃ is 4-halophenyl.

In certain other embodiments, the compound may be one of the following compounds

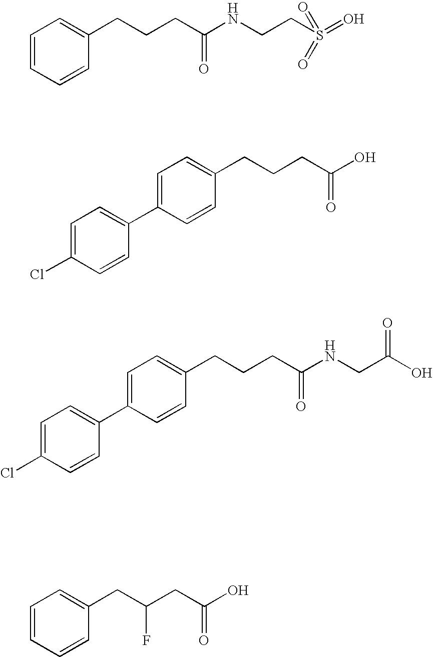

C. Tertiary Amine N-Oxides

In another aspect, the invention relates to compounds represented by Formula IV:

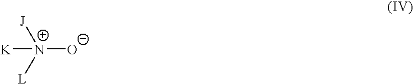

wherein
J, K, and L are independently for each occurrence alkyl, substituted alkyl, or —(CR₁R₂)ₙ-E-(CR₃R₄)ₘ—X;
X is H or CH₃;
R is independently for each occurrence H, lower alkyl, aryl, heterocyclyl;
R₁-R₄ are independently for each occurrence H, lower alkyl, fluoro or haloalkyl;
R₅ is independently for each occurrence H, lower alkyl, aryl, heterocyclyl;
m is independently for each occurrence 0-3;
n is independently for each occurrence 1-3;
E is O, S, SO, SO₂, —SO₂N(R₅)—, —N(R₅)SO₂—, NR₅, —C(O)O—, —O(O)C—, —CON(R₅)—, —N(R₅)CO—, or is absent;
   provided that when m=0, E is selected from O, NR₅R₆, CONR₅R₆, SO₂NR₅R₆; and R₆=H, lower alkyl, aryl, heterocyclyl;
   and pharmaceutically acceptable esters, salts, and prodrugs thereof. In certain preferred embodiments, the compound of the invention is not trimethylamine oxide or a compound represented by the formula:

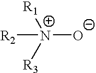

wherein

R₁, R₂, and R₃ are independently hydrogen, halogen, or lower C₁-C₆ alkyl;

or a pharmaceutically-acceptable salt thereof; or a mixture thereof.

Various preferred embodiments of this aspect of the invention include individual compounds of Formula IV wherein: at least one of J, K, and L is substituted alkyl; at least one of J, K, and L is an amino-substituted alkyl; at least one of J, K, and L is a methoxy-substituted alkyl.

Selected preferred compounds of the invention include: triethylamine oxide, (2-aminoethyl)dimethylamine oxide, (N′,N-dimethylaminoethyl)dimethylamine oxide, and (2-methoxyethyl)dimethylamine oxide.

The structures of some of the compounds of the invention, e.g., compounds of Formula I-IV, include asymmetric carbon atoms. Accordingly, the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. Uses of the Compounds of the Invention

Endoplasmic reticulum (ER) stress has been found to be important in the pathogenesis of a variety of diseases including al-anti-typsin deficiency, urea cycle disorders, type I diabetes, and cystic fibrosis. The present invention stems from the recognition that ER stress is implicated in the pathogenesis of diseases such as obesity, peripheral insulin resistance, hyperglycemia, and type 2 diabetes (Ozcan et al., “Endoplasmic Reticulum Stress Link Obesity, Insulin Action, and Type 2 Diabetes” Science 306:457-461, 2004; incorporated herein by reference), as well as hypercholesterolemia, atherosclerosis, and related conditions. In light of this discovery, agents that reduce or prevent ER stress have been shown herein to be useful in the treatment of obesity, insulin resistance, hyperglycemia, type 2 diabetes, as well as hypercholesterolemia, atherosclerosis, and related conditions.

Accordingly, in another aspect, the invention also provides methods for treating or preventing a condition in a subject related to ER stress, by administering to the subject an effective amount of a compound of the invention, e.g., a compound of Formulae I-IV or otherwise described herein. In certain embodiments, the invention provides methods treating a subject for hypercholesterolemia, atherosclerosis, and related conditions (or preventing the development of such conditions)

In certain embodiments, an agent known to reduce or modulate ER stress would be useful in treating these metabolic diseases. In this manner, and without wishing to be bound by theory, it is understood that these agents may act to reduce or prevent ER stress in any manner. In certain embodiments, the agent may increase the capacity of the ER to process proteins (e.g., increasing the expression of ER chaperones, increasing the levels of post-translational machinery). In other embodiments, the agent may reduce the quantity of proteins to be processed by the ER (e.g., decreasing the total level of protein produced in a cell, reducing the level of protein processed by the ER, reducing the level of mutant proteins, reducing the level of misfolded proteins). Yet other agents may cause the release of misfolded/mutant proteins from the ER. The agent may work in all cells, or the effect may be limited to certain cells type (e.g., secretory cells, epithelial cells, hepatocytes, adipocytes, endocrine cells, etc.). In certain embodiments, the agents are particularly useful in reducing ER stress in adipose cells. In other embodiments, the agents are particularly useful in reducing ER stress in hepatic cells. The agents may work on the transcriptional, translational, post-translational, or protein level to reduce or prevent ER stress.

Compounds useful for the treatment of disorders related to ER stress can be identified according a variety of methods, some of which are known in the art (see, e.g., PCT patent Publication WO2006031931, which is incorporated herein by reference).

In certain embodiments, a chemical compound or a collection of chemical compounds is assayed to identify compounds that reduce or modulate ER stress in vivo or in vitro. In certain screening assays, a test compound is contacted with a cell. The cell may be any type of cells with an endoplasmic reticulum; in certain embodiments, the cell is a mammalian cells, particularly a human cell. The cell may be derived from any organ system. In certain embodiments, the cell is a cell from adipose tissue or liver tissue. In some embodiments, a screening method for identifying an agent that reduce ER stress includes contacting a cell already experiencing ER stress with a candidate agent. The ER stress in the cell may be caused by any techniques known in the art. For example, ER stress may be due to a genetic alteration in the cells (e.g., XBP-I mutations) or the treatment with a chemical compound known to cause ER stress (e.g., tunicamycin, thapsigargin). The level of ER stress markers is assayed before and after addition of the test compound to determine if the compound reduces ER stress. Markers of ER stress that may be assayed include spliced forms of XBP-I, the phosphorylation status of PERK (e.g., Thr980), the phosphorylation status of eIF2α (e.g., Ser51), mRNA and/or protein levels of GRP78/BIP, and JNK activity. Test compounds that reduce the levels of ER stress markers may be useful for the reduction of ER stress in vitro or in vivo. As would be appreciated by one of skill in this art, the test compound may be tested at various concentrations and under various conditions (e.g., various cell types, various causes of ER stress (genetic vs. chemical), various formulations).

An example of an in vitro assay for screening for compounds useful for the treatment of ER stress-related disorders is described in more detail in Example 1, infra.

Animal models can also be used to identify compounds useful for treating conditions such as hypercholesteremia and atherosclerosis. For example, apoE−/− mice (a commonly used model of hypercholesterolemia and atherosclerosis) can be fed a western diet, with test animals receiving a dose of a test compound and control animals receiving a vehicle control. After a period of time (such as three months), the mice are sacrificed and aortas are dissected and fixed in 10% buffered formalin and stained with Oil-Red-O, which stains lipids. Test compounds which reduce the amount of Oil-Red-O staining compared to control may be useful in the prevention and treatment of the formation of atherosclerotic plaques.

Assays may also be used for identifying compounds that prevent ER stress. In screening for compounds that prevent ER stress, the cells are not experiencing ER stress before they are contacted with the test compound. After the cells are contacted with the test compound, an agent known to cause ER stress is added to the cells, and then the level of at least one ER markers is measured to determine whether the compound is able to prevent ER stress. As would be appreciated by one of skill in this art, the test compound may be tested at various concentrations and under various conditions.

Agents identified by a screening method may be further tested for toxicity, pharmacokinetic properties, use in vivo, etc. so that they may be formulated and used in the clinic to treat ER stress-related disorders such as obesity, type 2 diabetes, hyperglycemia, and insulin resistance. The identified agents may also find use in the treatment of other diseases such as hypercholesterolemia and atherosclerosis.

The administration of an effective dose of a compound of the present invention, or a combination therapy including a compound of the present invention, to a subject to treat or prevent obesity, insulin resistance, type 2 diabetes, hyperglycemia, hypercholesterolemia, atherosclerosis, or other related disease may cure the disease being treated, alleviate or reduce at least one sign or symptoms of the disease being treated, reduce the short term consequences of the disease, reduce the long term consequences of the disease, or provide some other transient beneficial effect to the subject. In certain embodiments, the inventive treatment increases insulin sensitivity. In other embodiments, the inventive treatment decreases blood glucose levels. In other embodiments, the inventive treatment prevents the long term consequences of diabetes including atherosclerosis, diabetic retinopathy, peripheral neuropathy, etc. In certain embodiments, the inventive treatment reduces levels of ER stress markers (e.g., spliced froms of XBP-1, phosphorylation status of PERK, phosphorylation of eIF2a, mRNA levels of GRP78B1P, protein levels of GRP78BIP, JNK activity) in cells (e.g., adipocytes, hepatocytes). In certain embodiments, the inventive treatment increases insulin action. In other embodiments, the inventive treatment increases insulin receptor signalling (e.g., phosphorylation of insulin receptor, IRS-1, IRS-2, akt). In certain embodiments, the inventive treatment suppresses appetite. In other embodiments, the inventive treatment prevents weight gain or promotes weight loss. In certain embodiments, the inventive treatment prevents the development of type 2 diabetes. In certain embodiments, the inventive treatment prevents the development of obesity. In certain embodiments, the inventive treatment prevents the development of hyperglycemia.

The agent may be combined with one or more other pharmaceutical agents, particularly agents traditionally used in the treatment of diabetes, obesity, or insulin resistance. A list of agents useful in combination with compounds of the invention (e.g., PBA, TUDCA, TMAO, or derivatives thereof) is provided in U.S. Provisional patent application Ser. No. 11/227,497. The list includes generic names, trade names, and manufacturers. Exemplary agents useful in combination with ER stress reducing agents include, but are not limited to, anti-diabetic agents (e.g. insulin, hypoglycemic agents (e.g., oral hypoglycemic agents such as sulfonylureas, tolbutamide, metformin, chlorpropamide, acetohexamide, tolazamide, glyburide, etc.)), anti-obesity agents, anti-dyslipidemia agent or anti-atherosclerosis agent (e.g., cholesterol lowering agents (e.g., HMg-CoA reductase inhibitors such as lovastatin, atorvastatin, simvastatin, pravastatin, fluvastatin, etc., aspirin), anti-obesity agent (e.g., appetite suppressants), vitamins, minerals, and anti-hypertensive agents.

In addition, one embodiment of the invention relates to the administration of an effective dose of compounds according to the invention, or a combination therapy including such compounds, to a subject to treat or prevent hypercholesterolemia, atherosclerosis, and related conditions may cure the disease being treated, alleviate or reduce at least one sign or symptoms of the disease being treated, reduce the short term consequences of the disease, reduce the long term consequences of the disease, or provide some other transient beneficial effect to the subject. In certain embodiments, the inventive treatment decreases blood cholesterol levels. In other embodiments, the inventive treatment prevents the long term consequences of hypercholesterolemia including atherosclerosis, stroke, and other ischemic vascular diseases, etc. In particular embodiments, the agent may be combined with one or more other pharmaceutical agents, particularly agents traditionally used in the treatment of hypercholesterolemia and atherosclerosis. Exemplary agents useful in combination with the compounds described herein include, but are not limited to, anti-dyslipidemia agents or anti-atherosclerosis agents (e.g., cholesterol lowering agents (e.g., HMg-CoA reductase inhibitors such as lovastatin, atorvastatin, simvastatin, pravastatin, fluvastatin, etc., aspirin), anti-obesity agent (e.g., appetite suppressants), vitamins, minerals, and anti-hypertensive agents.

In certain embodiments, the compound of the invention is used in combination with an anti-diabetic agent. Exemplary anti-diabetic agents include biguanides (e.g., metformin), sulfonylureas (e.g. glimepiride, glyburide, glibenclamide, glipizide, gliclazide), insulin and analogs thereof (e.g., insulin lispro, insulin glargine, exubera, AERx insulin diabetes management system, ATR inhaled insulin, oralin, insulin detemir, insulin glulisine), peroxisome proliferator-activated receptor-gamma agonists (e.g., rosiglitazone, pioglitazone, isaglitazone, rivoglitazone, T-131, MBX-102, R-483 CLX-0921), dual PPAR agonists and PPAR pan agonists (e.g., BMS-398585, tesaglitazar, muraglitazar, naveglitazar, TAK-559, netoglitazone, GW-677594, AVE-0847, LY-929, ONO-5129), combination therapies (e.g., metforminfglyburide, metformin/rosiglitazone, metformin, glipizide), meglitinides (e.g., repaglinide, nateglinide), alpha-glucosidase inhibitors (e.g., acarbose, miglitol, voglibose), glucagon-like peptide-1 (GLP-1) analogues and agonists (e.g., Exenatide, Exenatide LAR, Liraglutide, CJC-1131, AVE-0010, BIM-51077, N,N-2501, SUN-E7001), dipeptidyl peptidase IV (DPP-IV) inhibitors (e.g., LAF-237, MK-431 (Merck and Co), PSN-9301 (Probiodrug Prosidion), 815541 (GlaxoSmithKline-Tanabe), 823093 (GlaxoSmithKline), 825964 (GlaxoSmithKline), BMS-477118), pancreatic lipase inhibitors (e.g., orlistat), sodium glucose co-transporter (SGLT) inhibitors (e.g., T-1095 (Tanabe-J&J), AVE-2268, 869682 (GlaxoSmithKline-Kissei)), and amylin analog (e.g., pramlintide).

The invention provides systems and methods of treating type 2 diabetes, insulin resistance, obesity, and other related conditions that provide a better therapeutic profile than the administration of the ER stress modality or the other treatment modality alone.

In certain embodiments, a compound of the invention is used in combination with a hypoglycemic agent. For example, insulin, glucagon, a biguanide hypoglycemic agent (e g, metformin, phenformin, or buformin), a thiazolidinedione hypoglycemic agent (e.g., ciglitazone, pioglitazone), a sulfonylurea hypoglycemic agent (e.g, tolbutamide, chlorpropamide, acetohexamide, tolazamide, glyburide, glipizide, or gliclazide), an a-glucosidase inhibitor (e.g, acarbose), or diazoxide may be combined with glycerol, D₂O, dimethylsulfoxide (DMSO), 4-phenyl butyrate (PBA), tauroursodeoxycholic acid (TUDCA), glycine betaine (betaine), glycerolphosphocholine (GPC), methylamines, or trimethylamine N-oxide (TMAO), or any compound of this invention.

In certain embodiments, a compound of the invention is used in combination with an anti-obesity agent. Exemplary anti-obesity agents include pancreatic lipase inhibitors (e.g. orlistat), serotonin and norepinephrine reuptake inhibitors (e.g., sibutramine), noradrenergic anorectic agents (e.g., phentermine, mazindol), peripherally acting agents (e.g, ATL-962 (Alizyme), HMR-1426 (Aventis), GI 181771 (GlaxoSmithKline)), centrally acting agents (e.g, Recombinant human ciliary neurotrophic factor, Rimonabant (SR-141716) (Sanofi-Synthelabo), BVT-933 (GlaxoSmithKlineBiovitrum), Bupropion SR (GlaxoSmithKline), P-57 (Phytopharm)), thermogenic agents (e.g, TAK-677 (A.1-9677) (Dainippon/Takeda)), cannabinoid CB I antagonists (e.g., acomplia, SLV319), cholecystokinin (CCK) agonists (e.g, GI 181771 (GSK)), lipid metabolism modulator (e.g., AOD9604 (Monash University/Metabolic Pharmaceuticals), glucagon-like peptide I agonist (e.g, AC137 (Amylin)), leptin agonist (e.g., second generation leptin (Amgen), beta-3 adrenergic agonists (e.g, SR58611 (Sanofi-Aventis), CP 331684 (Pfizer), LY 377604 (Eli Lilly), n5984 (Nisshin Kyorin Pharmaceutical)), peptide hormone (e.g., peptide YY [3-36] (Nastech)), CNS modulator (e.g., S2367 (Shionogi & Co. Ltd.)), neurotrophic factor (e.g., peg axokine), and 5HT2C serotonin receptor agonist (e.g., APD356). Other anti-obesity agents include methamphetamine HCl, 1426 (Sanofi-Aventis), 1954 (Sanofi-Aventis), c-2624 (Merck & Co), c-5093 (Merck & Co), and T71 (Tularik).

In yet other embodiments, a compound of the invention is used in combination with an anti-dyslipidemia agent or anti-atherosclerosis agent. Exemplary anti-dyslipidemia agents or anti-atherosclerosis agents include HMG-CoA reductase inhibitors (e.g., atorvastatin, pravastatin, simvastatin, lovastatin, fluvastatin, cerivastatina, rosuvastatin, pitivastatin), fibrates (e.g., ciprofibrate, bezafibrate, clofibrate, fenofibrate, gemfibrozil), bile acid sequestrants (e.g, cholestyramine, colestipol, colesevelam), niacin (immediate and extended release), anti-platelets (e.g., aspirin, clopidogrel, ticlopidine), angiotensin-converting enzyme (ACE) inhibitors (e.g, ramipril, enalapril), angiotensin II receptor antagonists (e.g, losartan potassium), acyl-CoA cholesterol acetyltransferase (ACAT) inhibitors (e.g., avasimibe, eflucimibe, CS-505 (Sankyo and Kyoto), SW-797 (Sumito)), cholesterol absorption inhibitors (e.g, ezetimibe, pamaqueside), nicotinic acid derivatives (e g, nicotinic acid), cholesterol ester transfer protein (CETP) inhibitors (e.g, CP-529414 (Pfizer), JTT-705 (Japan Tobacco), CETi-1, torcetrapib), microsomal triglyceride transfer protein (MTTP) inhibitors (e.g., implitapide, R-103757, CP-346086 (Pfizer)), other cholesterol modulators (e.g., NO-1886 (Otsuka/TAP Pharmaceutical), CI-1027 (Pfizer), WAY-135433 (Wyeth-Ayerst)), bile acid modulators (e.g., GT102-279 (GelTex/Sankyo), HBS-107 (HisamitsuBanyu), BTG-511 (British Technology Group), BARI-1453 (Aventis), S-8921 (Shionogi), SD-5613 (Pfizer), AZD-7806 (AstraZeneca)), peroxisome proliferation activated receptor (PPAR) agonists (e.g., Tesaglitazar (AZ-242) (AstraZeneca), Netoglitazone (MCC-555) (Mitsubishi/Johnson & Johnson), GW-409544 (Ligand Pharmaceuticals/GlaxoSmithKline), GW-501516 (Ligand Pharmaceuticals/GlaxoSmithKline), LY-929 (Ligand Pharmaceuticals and Eli Lilly), LY-465608 (Ligand Pharmaceuticals and Eli Lilly), LY-518674 (Ligand Pharmaceuticals and Eli Lilly), MK-767 (Merck and Kyorin)), gene-based therapies (e.g, AdGVVEGF121.10 (GenVec), ApoA1 (UCB Pharma/Groupe Fournier), EG-004 (Trinam) (Ark Therapeutics), ATP-binding cassette transporter-A1 (ABCA1) (CV Therapeutics/Incyte, Aventis, Xenon)), composite vascular protectant (e.g. AGI-1067 (Atherogenics)), BO-653 (Chugai), glycoprotein IIb/IIIa inhibitors (e.g., Roxifiban (Bristol-Myers Squibb), Gantofiban (Yamanouchi), Cromafiban (Millennium Pharmaceuticals)), aspirin and analogs thereof (e.g., asacard, slow-release aspirin, pamicogrel), combination therapies (e.g., niacin/lovastatin, amlodipine/atorvastatin, simvastatin/lezetimibe), IBAT inhibitors (e.g., S-89-21 (Shionogi)), squalene synthase inhibitors (e.g., BMS-188494 I(Bristol-Myers Squibb), CP-210172 (Pfizer), CP-295697 (Pfizer), CP-294838 (Pfizer), TAK-475 (Takeda)), monocyte chemoattractant protein (MCP-1) inhibitors (e.g., RS-504393 (Roche Bioscience), other MCP-1 inhibitors (GlaxoSmithKline, Teijin, and Bristol-Myers Squibb)), liver X receptor agonists (e.g., GW-3965 (GlaxoSmithKline), TU-0901317 (Tularik)), and other new approaches (e.g., MBX-102 (Metabolex), NO-1886 (Otsuka), Gemcabene (Pfizer)).

In still other embodiments, a compound of the invention is used in combination with an anti-hypertensive agent. Examplary anti-hypertension agents include diurectics (e.g., chlorthalidone, metolazone, indapamide, bumetanide, ethacrynic acid, furosemide, torsemide, amiloride HCl, spironolactone, triamterene), alpha-blockers (e.g., doxazosin mesylate, prazosin HCl, terazosin HCl), betablockers (e.g., acebutolol, atenolol, betaxolol, bisoprolol fumarate, carteolol HCl, metoprolol tartrate, metoprolol succinate, nadolol, penbutolol sulfate, pindolol, propanolol HCl, timolol maleate, carvedilol), Ca²⁺ channel blockers (e.g., amlodipine besylate, felodipine, isradipine, nicardipine, nifedipine, nisoldipine, diltiazem HCl, verapamil HCl, azelnidipine, pranidipine, graded diltiazem formulation, (s)-amlodipine, clevidipine), angiotensin converting enzyme (ACE) inhibitors (e.g., benazepril hydrochloride, captopril, enalapril maleate, fosinopril sodium, lisinopril, moexipril, perindopril, quinapril hydrochloride, ramipril, trandolapril), angiotensin II (AT-II) antagonists (e.g., losartan, valsartan, irbesartan, candesartan, telmisartan, eprosartan, olmesarta, YM-358 (Yamanouchi)), vasopeptidase inhibitors (e.g. omapatrilat, gemopatrilat, fasidotril, sampatrilat, AVE 7688 (Aventis), MI00240 (Aventis), Z13752A (Zambon/GSK), 796406 (Zambon/GSK)), dual neutral endopeptidase and enotheline converting enzyme (NEP/ECE) inhibitors (e.g. SLV306 (Solvay), NEP inhibitors (e.g., ecadotril), aldosterone antagonists (e.g., eplerenone), renin inhibitors (e.g., Aliskiren (Novartis), SPP 500 (Roche/Speedel), SPP600 (Speedel), SPP 800 (Locus/Speedel)), angiotensin vaccines (e.g., PMD-3117 (Protherics)), ACE/NEP inhibitors (e.g., AVE-7688 (Aventis), GW-660511 (Zambon SpA)), Na⁺7⁺ ATPase modulators (e.g., PST-2238 (Prassis-Sigma-Tau), endothelin antagonists (e.g., PD-156707 (Pfizer)), vasodilators (e.g., NCX-4016 (NicOx), LP-805 (PolalWyeth)), naturetic peptides (e.g., BDNP (Mayo Foundation)), angiotensin receptor blockers (ARBs) (e.g., pratosartan), ACE crosslink breakers (e.g., alagebrium chloride), endothelin receptor agonists (e.g., tezosentan (Genentech), ambrisentan (Myogen), BMS 193884 (BMS), sitaxsentan (Encysive Pharmaceuticals), SPP301 (RochelSpeedel), Darusentan (MyogenlAbbott), 3104132 (Banyu/Merck & Co.), TBC3711 (Encysive Pharmaceuticals), SB 234551 (GSIUShionogi)), combination therapies (e.g., benazepril hydrochloride/hydrochlorothiazide, captopril/hydrochlorothiazide, enalapril maleate/hydrochlorothiazide, lisinopril/hydrochlorothiazide, losartan/hydrochlorothiazide, atenolol/chlorthalidone, bisoprolol fumarate/hydrochlorothiazide, metoprolol tartrate/hydrochlorothiazide, amlodipine besylate/benazepril hydrochloride, felodipine/enalapril maleate, verapamil hydrochloride/trandolapril, lercanidipine and enalapril, olmesartan/hydrochlorothiazide, eprosartan/hydrochlorothiazide, amlodipine besylate/atorvastatin, nitrendipine/enalapril), and MC4232 (University of Manitoba/Medicure).

In certain embodiments, a chemical chaperone (e.g., a compound of this invention) is used in combination with a vitamin, mineral, or other nutritional supplement.

In certain embodiments, a first compound (e.g., a compound described herein) is administered in a sub-optimal dose (e.g., an amount that does not manifest detectable therapeutic benefits when administered in the absence of a second agent). In such cases, the administration of such an sub-optimal dose of the first compound in combination with another agent results in a synergistic effect. The first compound and other agent work together to produce a therapeutic benefit. In other embodiments, the other agent (i.e., not the first compound) is administered in sub-optimal doses. In combination with the first compound, the combination exhibits a therapeutic effect. In yet other embodiments, both the first compound and the other agent are administered in sub-therapeutic doses, and when combined produce a therapeutic effect. The dosages of the other agent may be below those standardly used in the art.

The dosages, route of administration, formulation, etc. for anti-diabetic agents, anti-obesity agents, anti-dyslipidemia agent or anti-atherosclerosis agent, anti-obesity agent, vitamins, minerals, and anti-hypertensive agents (listed above) are known in the art. The treating physician or health care professional may consult such references as the Physician's Desk Reference (59^{th} Ed., 2005), or Mosby's Drug Consult and Interactions (2005) for such information. It is understood that a treating physician would exercise his professional judgment to determine the dosage regimen for a particular patient.

The invention provides systems and methods of treating hypercholesterolemia, atherosclerosis, and related conditions, that provide a better therapeutic profile than the administration of a compound described herein or the other treatment modality alone. In certain embodiments, the therapeutic effect may be greater. In certain embodiments, the combination has a synergistic effect. In other embodiments, the combination has an additive effect. The administration of a combination treatment regimen may reduce or even avoid certain unwanted or adverse side effects. In certain embodiments, the agents in the combination may be adminstered in lower doses, adminstered less frequently, or administered less frequently and in lower doses. Therefore, combination therapies with the above described benefits may increase patient compliance, improve therapy, and/or reduce unwanted or adverse side effects.

In certain embodiments, the subject is a mammal, in particular a human. In accordance with the methods of the invention, the compound can be administered in combination with a pharmaceutically acceptable carrier. In advantageous embodiments, the pharmaceutically-acceptable carrier provides sustained delivery of the compound to a subject for at least four weeks after administration to the subject.

In certain embodiments, the compound of the invention is administered orally. In other embodiments, the compound is administered intravenously. In yet other embodiments, the compound is administered topically. In still other embodiments, the compound is administered topically is administered parenterally.

Although dosages may vary depending on the particular indication, route of administration and subject, the compounds may be administered at a concentration of about 0.1 mg to about 1000 mg/kg of body weight.

Treatment can be initiated with smaller dosages which are less than the optimum dose of the compound. Thereafter, the dosage can be increased by small increments until the optimum effect under the circumstances is reached. For convenience, the total daily dosage may be divided and administered in portions during the day if desired. A therapeutically effective amount and a prophylactically effective amount of a compound of the invention is expected to vary from about 0.1 milligram per kilogram of body weight per day (mg/kg/day) to about 100 mg/kg/day.

The identification of those patients who are in need of prophylactic treatment or prevention for conditions related to ER stress, e.g., for hypercholesterolemia, atherosclerosis, and related conditions, is well within the ability and knowledge of one skilled in the art. Certain of the methods for identification of patients which are at risk of developing conditions or disorders related to ER stress, which may be treated by the subject method, are appreciated in the medical arts, such as family history of the development of a particular disease state and the presence of risk factors associated with the development of that disorder in the subject patient. A clinician skilled in the art can readily identify such candidate patients, by the use of, for example, clinical tests, physical examination and medical/family history.

### 4. Pharmaceutical Compositions

The invention also provides a pharmaceutical composition, comprising an effective amount a compound of the invention, e.g., a compound of Formulae I-IV or otherwise described herein, and a pharmaceutically acceptable carrier. In a further embodiment, the effective amount is effective to treat an ER-stress associated state, as described previously.

In a particular embodiment, the invention also provides a pharmaceutical composition, comprising an effective amount a compound of the invention, e.g., a compound of Formulae I-IV or otherwise described herein, and a pharmaceutically acceptable carrier, wherein the effective amount is effective to treat hypercholesterolemia, atherosclerosis, and related conditions, as described previously.

In an embodiment, the compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In certain embodiments, the subject is a mammal, e.g., a primate, e.g., a human.

The methods of the invention further include administering to a subject a therapeutically effective amount of a compound in combination with another pharmaceutically active compound, e.g., as described supra. Other pharmaceutically active compounds that may be used can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference. The compound of the invention and another pharmaceutically active compound may be administered to the subject in the same pharmaceutical composition or in different pharmaceutical compositions (at the same time or at different times).

The phrase “pharmaceutically acceptable” is refers to those compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (1₃) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound(s) of the invention include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a compound(s) with the carrier and, optionally, one or more accessory ingredients. In general, the formulations are prepared by uniformly and intimately bringing into association a compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound(s) include powders, sprays, ointments, pastes, creams, lotions, gels, solutions, patches and inhalants. The active compound(s) may be mixed under sterile conditions with a pharmaceutically-acceptable carrier, and with any preservatives, buffers, or propellants which may be required.

The ointments, pastes, creams and gels may contain, in addition to compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to compound(s) of the invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient.

A preferred dose of the compound for the present invention is the maximum that a patient can tolerate and not develop serious side effects. In certain embodiments, the compound of the present invention is administered at a concentration of about 0.001 μg to about 100 mg per kilogram of body weight, about 0.001-about 10 mg/kg or about 0.001 μg-about 1 mg/kg of body weight. Ranges intermediate to the above-recited values are also intended to be part of the invention.

### 5. Synthesis of Compounds of the Invention

Compounds of the invention can be synthesized by methods described in this section, and by methods generally known in the chemical literature. For example, compounds of the present invention can be prepared according to the following methods shown in the reaction schemes below.

A. Analogs of TUDCA

The present invention provides methods for the preparation of the novel analogues of TUDCA, and other endogeneous or non-endogeneous bile acids. Some of the novel compounds of this invention can be prepared using synthetic chemistry techniques well known in the art (see Comprehensive Organic Synthesis, Trost, B. M. and Fleming, I. eds., Pergamon Press, Oxford) to provide aryl- or heteroaryl-substituted butyric acid derivatives.

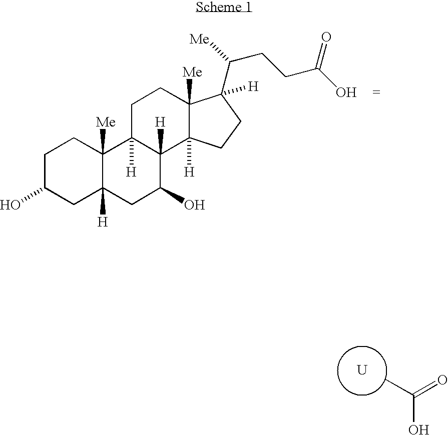

In one embodiment of the present invention analogues of tauroursodeoxycholic acid (TUDCA, Scheme 1) are synthesized. In Schemes 2-4 UCO₂H=UDCA (or alternative bile acid) and substituents such as R₁-R₅ correspond to, or yield, the substituents described in Formula I.

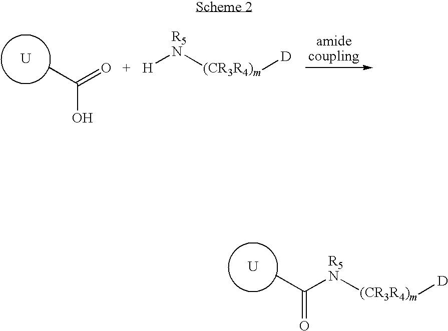

As shown in Scheme 2, UCO₂H may be coupled with a species HNR₅(CR₃R₄)ₘD under standard amide bond-forming conditions using techniques well known to those skilled in the art to provide a species UCOX as described previously. The product from the reaction in Scheme 2 can be isolated and purified employing standard techniques, such as solvent extraction, chromatography, crystallization, distillation and the like.

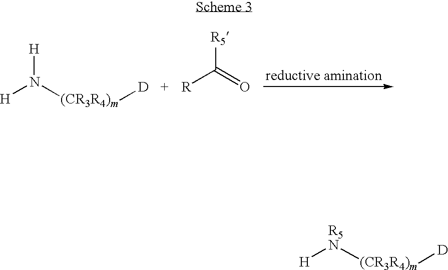

In a further embodiment the species HNR₅(CR₃R₄)ₘD may be synthesized according to the procedure illustrated in Scheme 3. Thus, H₂N(CR₃R₄)ₘD may be reacted with a species R₁₅COR under standard reductive amination conditions using techniques well known to those skilled in the art to provide HNR₅(CR₃R₄)ₘD. Typical reductive amination conditions employ a reducing agent such as sodium borohydride, sodium cyanoborohydride, or sodium triacetoxyborohydride and the like in suitable solvent (such as MeOH, EtOH, MeCN, DMN etc.) which is in contact with the reacting species at an appropriate temperature, typically in the range of −20° C. up to about 25° C. The reductive amination step may also be accomplished under standard hydrogenation conditions with H₂ (g) in the presence of a suitable catalyst such as Pd on carbon and the like. Further, the procedure in Scheme 3 may be accomplished in two steps by first reacting H₂N(CR₃R₄)ₘD with a species R₁₅COR under dehydrating conditions using techniques well known to those skilled in the art to provide an intermediate imine derivative. The intermediate imine may be carried on to the reduction step as a crude material or partially purified and then subjected to reductive conditions as described above. The product HNR₅(CR₃R₄)ₘD from the reaction in Scheme 3 can be isolated and purified employing standard techniques, such as solvent extraction, chromatography, crystallization, distillation and the like.

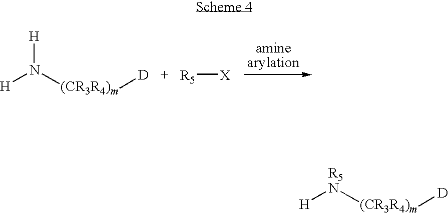

In another embodiment of the present invention the species HNR₅(CR₃R₄)ₘD may be synthesized according to the procedure illustrated in Scheme 4.

As shown in Scheme 4, H₂N(CR₃R₄)ₘD may be coupled with a species R₅ substituted with a group X. X maybe a metalloid species such as B(OR)₂, BiLn and the like and the reaction maybe promoted with stoichiometric or catalytic amounts of metal salts such as Cu(OAc)₂, CuI or CuOTf and the like. Typically, a base (e.g. pyridine, NEt₃, Cs₂CO₃, K₂CO₃ etc.) will also be present and the reaction carried out in a suitable solvent (e.g. CH₂Cl₂, THF, DME, toluene, MeCN, DMF, H₂O etc.). Additionally, molecular sieves may be used as a cocatalyst (see for example Fedorov, A. Y.; Finet, J-P. Tetrahedron Lett. 1999, 40, 2747-2748). Alternatively, X maybe a halogen or other functional group capable of undergoing a metal catalyzed N-arylation cross-coupling reaction. In which case, additional promoters such as 1,10-phenanthroline and dibenzylideneacetone may also be added to the reaction mixture. The cross-coupling reaction may be carried out at ambient temperature or heated to a temperature anywhere between about 30° C. to 150° C. The reaction mixture is then maintained at a suitable temperature for a time in the range of about 4 up to 72 hours, with 18 hours typically being sufficient. The product from the reaction can be isolated and purified employing standard techniques, such as solvent extraction, chromatography, crystallization, distillation and the like (see for example Lam, P. Y. S.; Clark, C. G.; Saubem, S.; Adams, J.; Winters, M. P.; Cham, D. M. T.; Combs, A. Tetrahedron Lett. 1998, 39, 2941-2944 and Kiyomori, A.; Marcoux, J. F.; Buchwald, S. L. Tetrahedron Lett. 1999, 40, 2657-2660).

In another embodiment of the present invention when X is a good aryl leaving group such as F, and R₅ is electron deficient or has one or more electron withdrawing substituents (e.g. NO₂, CN etc.), the coupling reaction may be effected thermally in a temperature range of about 60° C. up to about 250° C. Typically, this reaction is carried out in the presence of base (e.g. pyridine, NEt₃, Cs₂CO₃, K₂CO₃ etc.) in a suitable solvent, such as DMSO, DMF, DMA H₂O and the like, and takes from 1 h up to about 72 h with 18 hours typically being sufficient (see for example Russell, S. S.; Jahangir; Synth. Commun. 1994, 24, 123-130).

In addition, analogues of taurousodeoxycholic acid (TUDCA) can be prepared using other synthetic chemistry techniques well known in the art (see Comprehensive Organic Synthesis, Trost, B. M. and Fleming, I. eds., Pergamon Press, Oxford) and references cited therein. See also Coleman, J. P. et al., J. Steroid Biochem. Mol. Biol. (1998), 64(1-2), 91-101; Coleman, J. P. et al., J. Lipid Res. (1995), 36(4), 901-10; Parenti, M. PCT Publication No. WO 9218524; all of these references are incorporated herein by reference.

B. Analogs of PBA

The present invention provides methods for the preparation of the analogues of phenylbutyric acid (PBA). Some of the compounds of this invention can be prepared using synthetic chemistry techniques well known in the art (see Comprehensive Organic Synthesis, Trost, B. M. and Fleming, I., eds., Pergamon Press, Oxford) to provide aryl- or heteroaryl-substituted butyric acid derivatives. In general, the product from the reactions described herein can be isolated and purified employing standard techniques, such as solvent extraction, acid-base extraction, chromatography, crystallization, distillation and the like.

In the Schemes below the substituents A, B and D are the same as in Formula I except where defined otherwise. Substituents such as R₁ and R₂ are clear from context to correspond to, or to yield, the substituents described in Formula I.

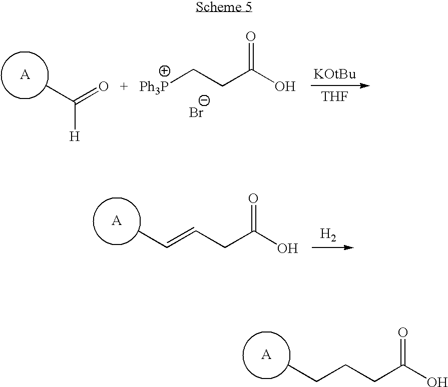

Thus in Scheme 5, an aryl or heteroaryl moiety A with a carboxaldehyde substituent (either obtained from a commercial source or prepared using synthetic chemistry techniques well known in the art) is reacted with (2-carboxyethyl)triphenylphosphonium bromide in a suitable solvent (e.g. THF, DME, DMF, Et₂O, tBuOH, etc., or mixtures thereof) in the presence of a base, such as potassium tert-butoxide or sodium hydride and the like at a temperature from 0° C. to 100° C., with 25° C. being presently preferred, for a sufficient period of time (typically about 2 to 18 h) to form a substituted alkene derivative (see for example Fujita, M. et al. Bioorg. Med. Chem. Lett. 2002, 12, 771-774). The resulting alkene derivative is then hydrogenated under the appropriate conditions (e.g. H₂ (g), catalytic Pd/C; or diimide; or LiAlH₄; or NaHTe; or Wilkinson's catalyst) in a suitable solvent to provide a PBA analogue as shown.

As shown in Scheme 6, the PBA analogue from Scheme 1 may then be coupled with a species HNR₅(CR₃R₄)ₘD under standard amide bond-forming conditions to provide a species A-B-D of Formula I as described above.

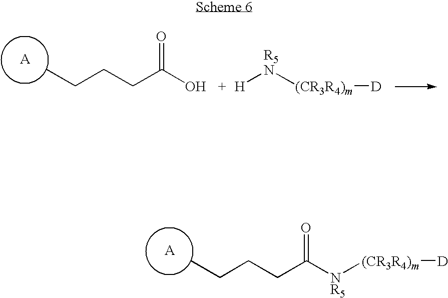

In another embodiment of the invention, the compounds of the present invention can be prepared as shown in Scheme 7 below.

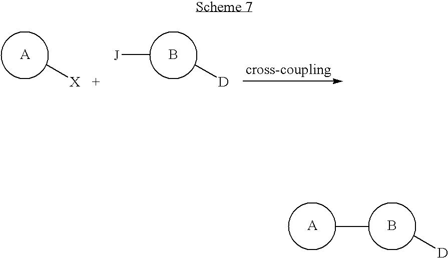

In Scheme 7, A, B and D are as described for Formula I and X is a halogen, trifluoromethanesulfonate (triflate) or similarly reactive species. Thus, A-X in Scheme 7 is reacted with a species J-B-D under metal-catalyzed cross-coupling conditions where J is a metallic or metalloid species such as B(OR)₂, Li, MgHal, SnR₃, ZnHal, SiR₃ and the like which is capable of undergoing a metal-catalyzed cross-coupling reaction. The coupling may be promoted by a homogeneous catalyst such as Pd(PPh₃)₄ or PdCl₂(PPh₃)₂, or by a heterogeneous catalyst such as Pd on carbon in a suitable solvent (e.g. THF, DMF, toluene, MeCN, DMF, H₂O etc.). Typically a base, such as K₂CO₃, NEt₃, and the like, will also be present in the reaction mixture. Other promoters may also be used such as CsF. The coupling reaction is typically allowed to proceed by allowing the reaction temperature to warm slowly from about 0° C. up to ambient temperature over a period of several hours. The reaction mixture is then maintained at ambient temperature, or heated to a temperature anywhere between about 30° C. to 150° C. The reaction mixture is then maintained at a suitable temperature for a time in the range of about 4 up to 48 hours, with about 18 hours typically being sufficient (see for example Miyaura, N.; Suzuki, A. Chem. Rev. 1995, 95, 2457-2483). The product from the reaction can be isolated and purified employing standard techniques, such as solvent extraction, acid-base extraction, chromatography, crystallization, distillation and the like.

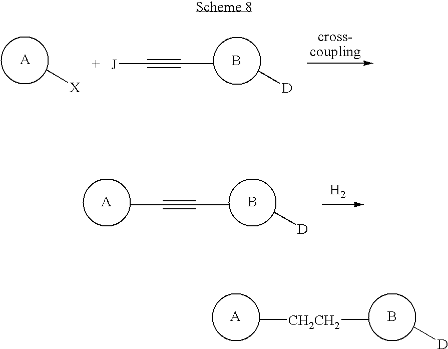

In Scheme 8 A, B and D are as described previously and X is a halogen, trifluoromethanesulfonate (triflate) or similarly reactive species. Thus, A-X in Scheme 4 is reacted with a species J-B-D containing a reactive alkyne under metal-catalyzed cross-coupling conditions where J is hydrogen, or a metallic or metalloid species such as B(OR)₂, Li, MgHal, SnR₃, ZnHal, SiR₃ and the like which is capable of undergoing a metal-catalyzed, cross-coupling reaction. The coupling may be promoted by a homogeneous catalyst such as Pd(PPh₃)₄ or PdCl₂(PPh₃)₂, or by a heterogeneous catalyst such as Pd on carbon in a suitable solvent (e.g. THF, DME, toluene, MeCN, DMF, H₂O etc.). Typically a co-catalyst such as copper (I) iodide and a base, such as K₂CO₃, NEt₃, and the like, will also be present in the reaction mixture. Other promoters may also be used such as CsF. The coupling reaction is typically allowed to proceed by allowing the reaction temperature to warm slowly from about 0° C. up to ambient temperature over a period of several hours. The reaction mixture is then maintained at ambient temperature, or heated to a temperature anywhere between about 30° C. to 150° C. The reaction mixture is then maintained at a suitable temperature for a time in the range of about 4 up to 48 hours, with about 18 hours typically being sufficient (see for example Bleicher, L. S. et al. J. Org. Chem. 1998, 63, 1109-1118). The resulting alkyne is then hydrogenated under the appropriate conditions (e.g. H₂ (g), catalytic Pd/C; or diimide; or LiAlH₄; or NaHTe; or Wilkinson's catalyst) in a suitable solvent to provide a PBA analogue as shown. The product from the reaction can be isolated and purified employing standard techniques, such as solvent extraction, acid-base extraction, chromatography, crystallization, distillation and the like.

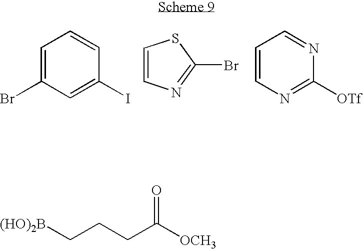

Examples of reactive species that might each be employed in Schemes 7 and 8 are shown in Scheme 9. Thus, A-X could be 1-bromo-3-iodobenzene which would undergo a cross-coupling reaction with a species J-B-D, coupling at the more reactive carbon-iodine position, to form a substituted bromobenzene derivative. This compound could then be further modified by cross-coupling at the carbon-bromine position to provide a PBA analogue substituted on the phenyl ring. In addition, A-X could be selected from 2-bromothiazole (see Feuerstein, M. et al. Tetrahedron Lett. 2005, 46, 1717-1720) or pyrimidin-2-yl trifluoromethanesulfonate (see Sandosham, J; Undheim, K. Heterocycles, 1994, 37, 501-514) to form substituted or unsubstituted heteroaryl analogues of PBA. Again, in Scheme 7, the species J-B-D might be 4-methoxy-4-oxobutylboronic acid, prepared according to the method of Falck (Falck, J. R. et al. Tetrahedron Lett. 2001, 42, 7211-7212).

Fluorinated derivatives of PBA and its analogues may be synthesized using techniques well known to those skilled in the art. For example, Scheme 10 illustrates one method used by Okano and coworkers to synthesize 2,2-difluoro-4-phenylbutanoic acid (see Okano, T. et al. Tetrahedron 1995, 51, 1903-1920). Thus, in Scheme 10, photolysis of 2-thioxopyridin-1(2H)-yl-3-phenylpropanoate in the presence of 1,1-dichloro-2,2-difluoroethene provides 1,1-dichloro-2,2-difluoro-1-(2-pyridylthio)-4-phenylbutane. This material is then heated under reflux with silver nitrate in a THE water mixture to give the difluorinated PBA analogue 2,2-difluoro-4-phenylbutanoic acid.

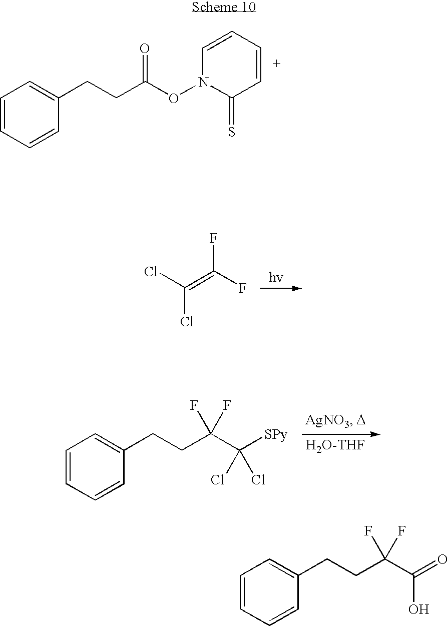

Another method for synthesizing fluorinated compounds was reported by Buss (Buss, C. W. et al. J. Fluorine Chem. 1986, 34, 83-104) and is illustrated in Scheme 11. Thus, commercially available ethyl 3-oxo-4-phenylbutanoate is treated with diethylaminosulfur trifluoride (DAST) in benzene to give (E)-ethyl-3-fluoro-4-phenylbut-3-enoate and ethyl-3,3-difluoro-4-phenylbutanoate as shown in Scheme 7. (E)-Ethyl-3-fluoro-4-phenylbut-3-enoate could then be hydrogenated and saponified to give the monofluorinated PBA analogue ethyl-3-fluoro-4-phenylbutanoic acid, and 3,3-difluoro-4-phenylbutanoate could be saponified to give the difluorinated PBA analogue 3,3-difluoro-4-phenylbutanoic acid.

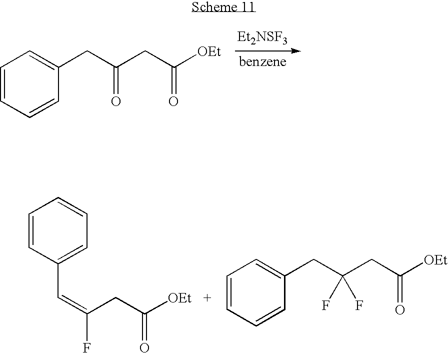

In addition, many of the analogues of phenylbutyric acid (PBA) described above can be prepared using other synthetic chemistry techniques well known in the art (see Comprehensive Organic Synthesis, Trost, B. M. and Fleming, I. eds., Pergamon Press, Oxford) and references cited therein.

C. Tertiary Amine N-Oxides

In general, tertiary amine N-oxides can be prepared by oxidation of the corresponding tertiary amine. Tertiary amines can be purchased from commercial sources or can be prepared according to a variety of methods well known in the art.

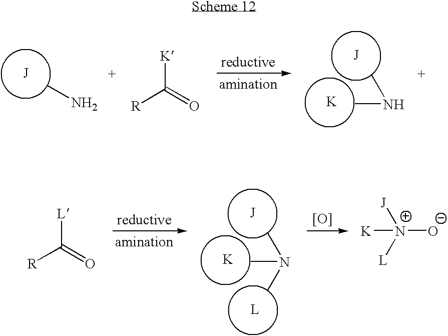

For example, compounds of the present invention (generally represented by the structure JKLN⁺O⁻ in which J, K and L are as described in Formula I) may be synthesized according to the procedures illustrated in Scheme 12. Thus, for example, the species JNH₂ may be reacted with an aldehyde or ketone K′COR under standard reductive amination conditions to provide a secondary amine of formula JKNH. Typical reductive amination conditions employ a reducing agent such as sodium borohydride, sodium cyanoborohydride, or sodium triacetoxyborohydride and the like in suitable solvent (such as MeOH, EtOH, MeCN, DMF etc.) which is in contact with the reacting species at an appropriate temperature, typically in the range of −20° C. up to about 25° C. Alternatively, the reductive amination may be accomplished under standard hydrogenation conditions with H₂ (g) in the presence of a suitable catalyst such as Pd on carbon and the like. The same procedure may be employed in the next step shown in Scheme 12 by reacting the secondary amine JKNH with an aldehyde or ketone L′COR, followed by reduction to give the tertiary amine JKLN.

Reductive amination may also be accomplished by reacting an amine (e.g., H₂NJ) with a carbonyl-containing species (e.g., K′COR) under dehydrating conditions using techniques well known to those skilled in the art to provide an intermediate imine derivative. The intermediate imine may be carried on to the reduction step as a crude material or partially purified and then subjected to reductive conditions as described above. Finally, the tertiary amine JKLN is oxidized using methods well known to those skilled in the art, using oxidizing reagents such as H₂O₂, tBuOOH or MCPBA and the like in a suitable solvent at an appropriate temperature. The products JKLN and JKLN⁺O⁻ from the reaction in Scheme 12 can be isolated and purified employing standard techniques, such as solvent extraction, chromatography, crystallization, distillation and the like.

In addition, many of the analogues of trimethylamine oxide acid (TMAO) described above can be prepared using other synthetic chemistry techniques well known in the art (see Comprehensive Organic Synthesis, Trost, B. M. and Fleming, I. eds., Pergamon Press, Oxford,) and references cited there within.

### EXEMPLIFICATION OF THE INVENTION

The present invention may further be exemplified using the following non-limiting examples.

### Example 1

### ERSE/URSE Activity Assay

The following assay may be used to identify compounds that will be useful in treating conditions related to ER stress.

When ER stress is triggered, a number of signaling events called unfolded protein response (UPR) is activated. One of the downstream effects of UPR is to increase transcriptional activity of genes with the promoter regions containing the endoplasmic reticulum stress element (ERSE) and unfolded response element (UPRE). One of UPR's eventual goals is to make more of those chaperone proteins needed for protein folding and destruction and thereby to increase functional capacity.

Accordingly, the compounds of the present invention may be analysed using this phenomena. For example, the results of screening test compounds may be expressed as percent induction of the ERSE reporter gene relative to tunicamycin treatment. Moreover, analysis may be made on ratios of reporter activity (firefly luciferase)/pCMV-RL (renilla luciferase) with basal reporter activity subtracted out, e.g., measuring increasing ER functional capacity. Alternatively, test compounds may be analyzed using percent repression of the tunicamycin induction of the ERSE or UPRE reporter gene. Further analysis may be made using the ratios of reporter activity (firefly luciferase)/pCMV-RL (renilla luciferase) with basal reporter activity subtracted out, e.g., measuring how well these compounds suppress ER stress. Negative numbers mean additional activation on top of tunicamycin activation.

Greater than 70 compounds of the invention were screened using such assays and most were found to be active according to this assay.

### Assay Details

### Cells

All transient transfections are done in HEK293 cells (obtained from DSMZ (German Collection of Microorganisms and Cell Cultures), Braunschweig Germany; DSMZ no.: ACC 305) grown in Minimum essential medium (Eagle) with 2 mM L-glutamine and Earle's BSS supplemented with 10% fetal bovine serum, 2 mM glutamine, 0.1 mM non-essential amino acids, 1 mM sodium pyruvate, Penicillin/Streptamycin respectively, at 37° C. in 5% CO2.

Cells are subcultured every 3 days by splitting a confluent culture 1:12.

### Culture Media

Minimum Essential Medium (EM) with Earle's Salts, without L-Glutamine Minimum Essential Medium (MEM) with Earle's Salts, without L-Glutamine, without Phenol Red GlutaMAX™-I Supplement, 200 mM (Invitrogen #35050038)

### MEM Non Essential Amino Acids (100×) (Invitrogen #11140035)

### Sodium Pyruvate MEM 100 mM (Invitrogen #1.1360039)

### Penicillin-Streptomycin Solution (10000:10000) (Invitrogen #15140122)

### Trypsin-EDTA (0.05% Trypsin, 0.53 mM EDTA•4Na) (Invitrogen #25300054)

### FCS (PAA Laboratories #A15-649)

charcoal-dextran treated FBS (Hyclone/pb Perbio # SH30068.03)

### Transient Transfection in 96 Well Plates

plates
BD/Falcon #353947 96-well White TC plates with clear Polystyrene Optilux bottom
or
Corning B.V/costar #3903 96 well cell culture plate, white, clear bottom
transfection reagent

### FuGENE 6 (Roche Applied Sciences # 11 988 387 001)

lysis buffer: 1× Passive Lysis Buffer (Promega # E1941)

### Day 1:

Inoculate 5×10⁴ HEK293 cells in 100 μl complete medium without phenol red and 10% charcoal-dextran-stripped FCS into white 96 well plates with transparent bottom and grow O/N (12-16 h)

### Day 2:

### Transfection

Cells should be approximately 80% confluent at time of transfection

prepare transfection mix in OptiMEM (Invitrogen #31985-047)

DNA:FuGENe 6 ratio=1:6 (v/v); a minimum of 100 ng DNA/well
DNA per well: 50 ng reporter (5×UPRE or 2×ERSE), 50 ng carrier DNA (sheared herring sperm DNA (Roche #223 646), 0.3 ng pCMV-RL, 0.25 ng pTREX-NTCP
1. Mix FuGENE 6 solution with OptiMEM, 10 μl per well
2. Mix OptiMEM with DNA, 10 μl per well
3. Add FuGENE 6 solution to DNA solution, mix immediately

### 4. Incubate 15-30 min at RT

add 20 ul transfection mix per well to cells in 100 μl medium

duration: minimum 4 h

### Medium Change

After 4 h: medium including transfection mix is shaked off the cells/plates and liquid droplets adhering to the plates wiped off quickly on a paper towel. This just leaves an even very thin liquid film on the cell layer. Immediately add carefully 100 μl of fresh pre-warmed medium including 1% charcoal-dextran-stripped FCS.

### Compound Addition

Final top concentrations of compounds were prediluted in complete medium without phenol red and 1% charcoal-dextran-stripped FCS and 2 fold dilutions prepared with the help of a pipetting roboter in medium+1% charcoal-dextran-stripped FCS and an identical amount of vehicle as for the final top concentration. All dilutions thus contain identical vehicle concentrations.
Compound stocks were either prepared in OptiMEM plus 50 mM HEPES pH7.0 (in some cases traces of NaOH were added to facilitate solution) or in DMSO. Final DMSO concentration was 0.1%.
12.5 μl compound solution per well was added 1-2 h after medium change.

### Tunicamycin Addition

1-2 h after compound addition tunicamycin (Sigma #93755; 10 mg/ml stock in DMSO, stored in aliquots at −20° C.) is added in 12.5 μl medium as above to give 0.2 μg/ml final concentration/well (all final concentrations are calculated for 125 μl total medium/well).

### Day 3:

Lysis—20 h after tunicamycin addition

remove medium, add 20 μl lysis buffer per well

shake vigorously 15 min

measure directly the whole extract within the culture plate or freeze extracts (extracts can be stored at 4° C. for some hours with slight loss in activity, but should be prewarmed to RT before measurement)

### Dual Luciferase Measurement

Assay buffers can either be commercially available buffers like Promega #E1910 or others or can be selfinade.
Firefly and Renilla luciferase activities were measured sequentially in the same cell extract using a BMG LABTECH LUMIstar OPTIMA luminometer and 75 μl of each assay buffer.
Measurement values are expressed as RLU for the primary reporter enzyme firefly luciferase (“FF”) or firefly RLU normalised by the values for the control reporter renilla luciferase (Ren: FF/Ren*1000).

### Further Compound Screening

In addition, compounds that show positive results in the ERSE/URSE Activity Assay are further screened using the analytical levels of ER stress markers. Examples of markers of ER stress include spliced forms of XBP-1, the phosphorylation status of PERK (Thr980) and eIF2a (Ser51), mRNA and protein levels of GRP78BIP, and JNK activity. Agents that when contacted with a cell with ER stress cause a reduction in the markers of ER stress as compared to an untreated control cell are identified as agents that reduce ER stress. A decrease in the levels of an ER stress marker are indicative of an agent that is useful in treating diseases associated with ER stress, such as obesity, type 2 diabetes, insulin resistance, hyperglycemia, cystic fibrosis, and Alzheimer's diseases. Agents identified using the inventive method are part of the invention.

### Example 2

Soft Gelatin Capsule Formulation I

| Item | Ingredients | mg/Capsule |
|---|---|---|
| 1. | Compound | 10.001-0.02 |
| 2. | Butylated Hydroxytoluene (BHT) | 0.016 |
| 3. | Butylated Hydroxyanisole (BHA) | 0.016 |
| 4. | Miglyol 812 qs. | 160.0 |

### Manufacturing Procedure:

1. BHT and BHA is suspended in Miglyol 812 and warmed to about 50° C. with stirring, until dissolved.

2. A compound of the invention is dissolved in the solution from step 1 at 50° C.

3. The solution from Step 2 is cooled at room temperature.

4. The solution from Step 3 is filled into soft gelatin capsules.

Note: All manufacturing steps are performed under a nitrogen atmosphere and protected from light.

### Example 3

Soft Gelatin Capsule Formulation II

| Item | Ingredients | mg/Capsule |
|---|---|---|
| 1. | Compound of the invention | 10.001-0.02 |
| 2. | di-α-Tocopherol | 0.016 |
| 3. | Miglyol 812 qs. | 160.0 |

### Manufacturing Procedure:

1. Di-α-Tocopherol is suspended in Miglyol 812 and warmed to about 50° C. with stirring, until dissolved.

2. A compound of the invention.

3. The solution from Step 2 is cooled at room temperature.

4. The solution from Step 3 is filled into soft gelatin capsules.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A compound represented by Formula I: 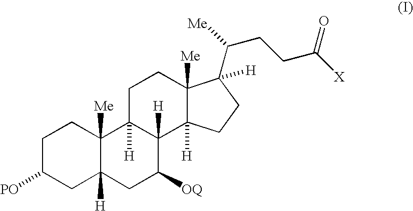
in which
P and Q are independently for each occurrence H, lower alkyl, PO₃R₂, SO₃R or COR;
X is OR, NRₐR_{b} or NR—B-D;
B is alkylene, substituted alkylene, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″, or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R is H, lower alkyl, aryl, or heterocyclyl;
R₁-R₄ are independently for each occurrence H, lower alkyl, fluoro or haloalkyl;
R₅ is H, lower alkyl, or substituted or unsubstituted aryl, or substituted or unsubstituted heteroaryl;
m is 1-3; and
n is 0-3;
and pharmaceutically acceptable esters, salts, and prodrugs thereof;
provided that the compound is not a compound represented by the formula: 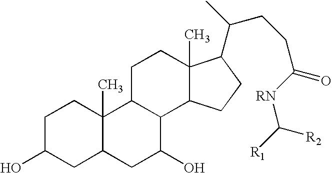
wherein
R is —H or C₁-C₄ alkyl;
R₁ is CH₂—SO₃R₃ and R₂ is —H; or R₁ is —COOH and R₂ is —CH₂—CH₂—CONH₂, —CH₂—CONH₂, —CH₂—CH₂—SCH₃ or —CH₂—S—CH₂—COOH; and
R₃ is —H or a basic amino acid; or a pharmaceutically acceptable salt thereof;
and provided that the compound is not one of a compound selected from the group consisting of 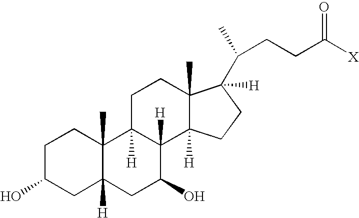
in which X is one of the following moieties:

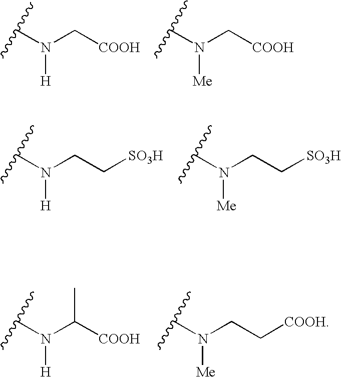

2. The compound of claim 1, in which P and Q are each H; X is NR—B-D; B is alkylene or substituted alkylene; D is CO₂R, CONRₐR_{b}, or PO₃RₐR_{b}; and R is H or lower alkyl.

3. The compound of claim 1 represented by the formula: 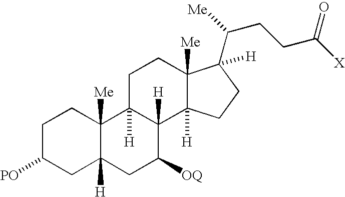
in which X is: 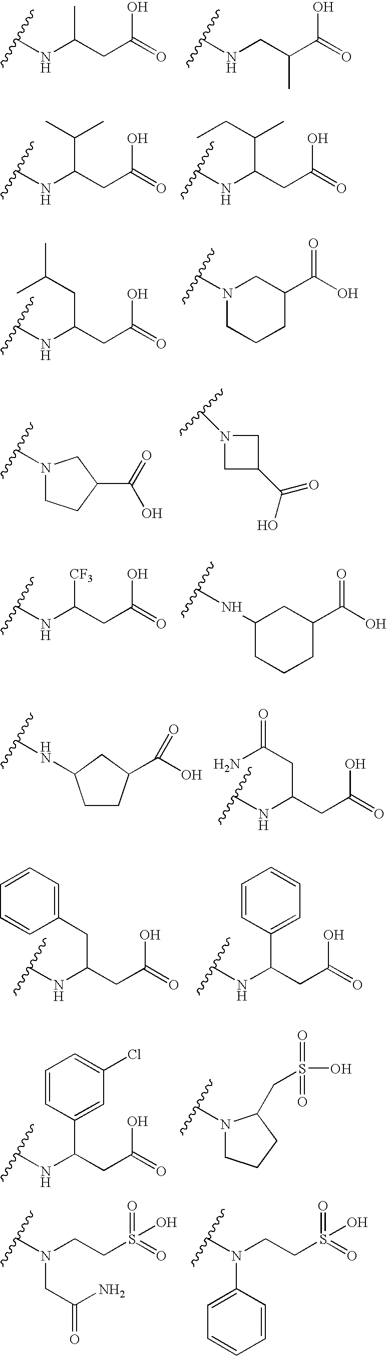 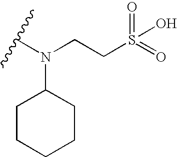
and pharmaceutically acceptable esters, salts, and prodrugs thereof.

4. A method for treating or preventing a condition related to ER-stress in a subject in need thereof, comprising administering to said subject an effective amount of a compound of claim 1, such that the condition related to ER-stress is treated or prevented in said subject.

5. The method of claim 4, wherein said condition related to ER-stress is selected from the group consisting of obesity, insulin resistance, type 2 diabetes, hyperglycemia, hypercholesterolemia, atherosclerosis and other ER-stress related diseases.

6. The method of claim 4, further comprising identifying a subject in need of prevention or treatment for ER stress-related diseases or conditions.

7. The method of claim 4, further comprising the step of obtaining the compound of Formula I.

8. The method of claim 4, wherein the subject is a mammal.

9. The method of claim 4, in which the subject is a human.

10. A pharmaceutical composition, comprising an effective amount of a compound of claim 1 and a pharmaceutically acceptable carrier.

11. The pharmaceutical composition of claim 10, wherein said effective amount is effective to treat an ER-stress associated state.

12. (canceled)

13. A packaged formulation comprising a pharmaceutical composition comprising a compound according to claim 1, and instructions for use in the treatment of an ER-stress associated state.

14. The packaged formulation of claim 13, wherein said compound is present in an amount effective to treat the ER-stress associated state.

15. The packaged formulation of claim 10, wherein said ER-stress associated state is a disorder selected from the group consisting of obesity insulin resistance, type 2 diabetes hyperglycemia, hypercholesterolemia, atherosclerosis and related diseases.

16. A compound represented by Formula II:
A-B-D (II)
wherein:
A is substituted or unsubstituted aryl, or substituted or unsubstituted heteroaryl;
B is alkyl, substituted alkyl, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″ or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R₁-R₄ are independently for each occurrence H, halogen, lower alkyl, or haloalkyl;
R₅ is H, lower alkyl, or substituted or unsubstituted aryl, or substituted or unsubstituted heteroaryl;
m is 1-3; and
n is 0-3;
and pharmaceutically acceptable esters, salts, and prodrugs thereof;
provided that the compound is not a compound represented by the following formula: 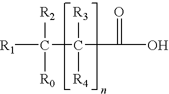
wherein
n is 1 or 2;
R₀ is aryl, heteroaryl, or phenoxy, the aryl and phenoxy being unsubstituted or substituted with, independently, one or more halogen, hydroxy or lower alkyl;
R₁ and R₂ are independently H, lower alkoxy, hydroxy, lower alkyl or halogen; and
R₃ and R₄ are independently H, lower alkyl, lower alkoxy, or halogen; or
a pharmaceutically-acceptable derivative or salt thereof.

17-24. (canceled)

25. The compound of claim 16 represented by Formula IIa or IIb: 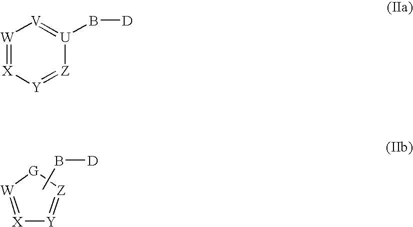
wherein:
U is C or N;
V, W, X, Y, and Z are independently for each occurrence CR₆, NR₇, O, or S;
G is NR₇, O, or S;
R₆ is independently for each occurrence H, halogen, alkyl, aryl, or heterocyclyl;
R₇ is H, alkyl, aryl, or heterocyclyl, or is absent;
B is alkyl, substituted alkyl, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″ or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R₁-R₄ are independently for each occurrence H, halogen, lower alkyl, or haloalkyl;
R₅ is H, lower alkyl, or substituted or unsubstituted aryl, or substituted or unsubstituted heteroaryl;
m is 1-3; and
n is 0-3;
and pharmaceutically acceptable esters, salts, and prodrugs thereof.

26. (canceled)

27. The compound of claim 16 represented by Formula IIa or IIIb: 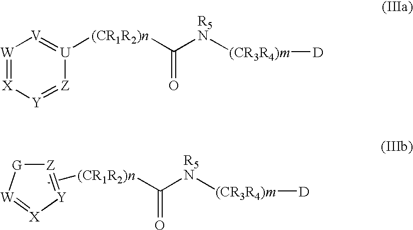
wherein
U is C or N;
V, W, X, Y, and Z are independently for each occurrence CR₆, NR₇, O, or S;
G is NR₇, O, or S;
R₆ is independently for each occurrence H, halogen, alkyl, aryl, or heterocyclyl;
R₇ is H, alkyl, aryl, or heterocyclyl, or is absent;
D is CO₂R, CONRₐR_{b}, SO₃R, SO₂NRₐR_{b}, tetrazolyl, —B(OR)(OR″), —P(O)R′OR″ or PO₃R′R″;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R₁-R₄ are independently for each occurrence H, halogen, lower alkyl, or haloalkyl;
R₅ is H, lower alkyl, or substituted or unsubstituted aryl, or substituted or unsubstituted heteroaryl;
m is 1-3; and
n is 0-3.

28. (canceled)

29. (canceled)

30. The compound of claim 27 selected from the group consisting of:

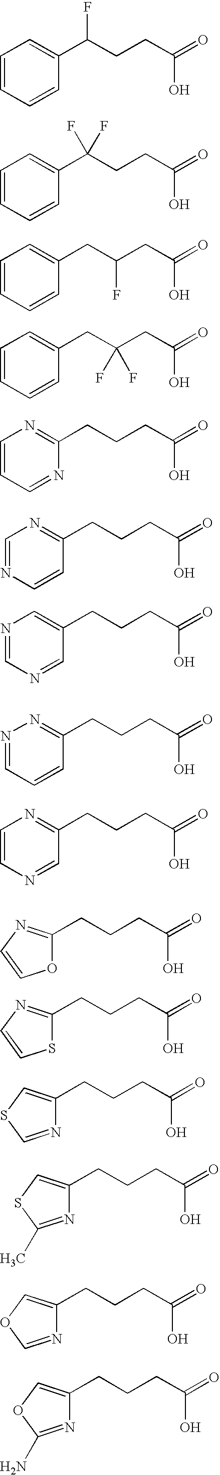

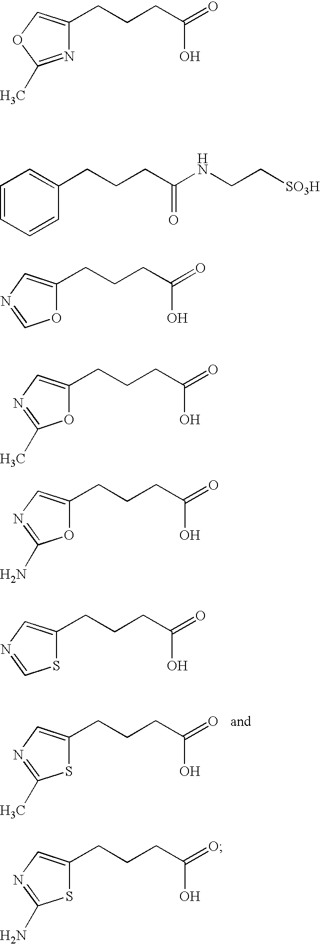

31. A method for treating or preventing a condition related to ER-stress in a subject in need thereof, comprising administering to said subject an effective amount of a compound of claim 16, such that condition related to ER-stress is treated or prevented in said.

32-36. (canceled)

37. A pharmaceutical composition, comprising an effective amount of a compound of claim 16 and a pharmaceutically acceptable carrier.

38. (canceled)

39. (canceled)

40. A packaged formulation comprising a pharmaceutical composition comprising a compound recited in claim 16, and instructions for use in the treatment of an ER-stress associated state.

41. (canceled)

42. (canceled)

43. A method for treating or preventing a condition related to ER-stress in a subject in need thereof, comprising administering to said subject an effective amount of a compound represented by Formula I: 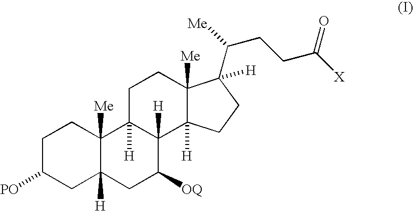
in which
P and Q are independently for each occurrence H, lower alkyl, PO₃R₂, SO₃R or COR;
X is OR, NRₐR_{b} or NR—B-D;
B is alkylene, substituted alkylene, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″, or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R is H, lower alkyl, aryl, or heterocyclyl;
R₁-R₄ are independently for each occurrence H, lower alkyl, fluoro or haloalkyl;
R₅ is H, lower alkyl, or substituted or unsubstituted aryl, or substituted or unsubstituted heteroaryl;
m is 1-3; and
n is 0-3;
and pharmaceutically acceptable esters, salts, and prodrugs thereof;
such that said condition is treated or prevented;
provided that the compound is not a compound represented by the formula: 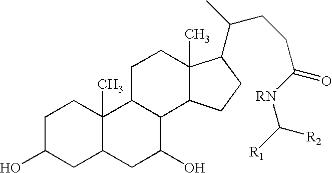
wherein
R is —H or C₁-C₄ alkyl;
R₁ is CH₂—SO₃R₃ and R₂ is —H; or R₁ is —COOH and R₂ is —CH₂—CH₂—CONH₂, —CH₂—CONH₂, —CH₂—CH₂—SCH₃ or —CH₂—S—CH₂—COOH; and
R₃ is —H or a basic amino acid; or a pharmaceutically acceptable salt thereof.

44. A method for treating or preventing a condition related to ER-stress in a subject in need thereof, comprising administering to said subject an effective amount of a compound represented by the formula: 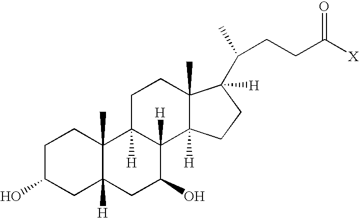
in which X is one of the following moieties: 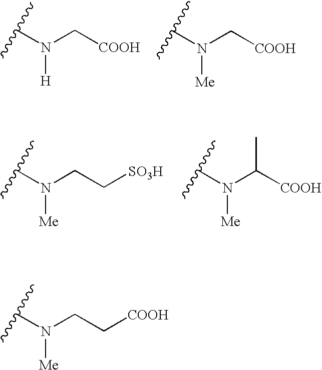
and pharmaceutically acceptable esters, salts, and prodrugs thereof;
such that said condition is treated or prevented.

45. The method of claim 43, wherein said condition related to ER-stress is selected from the group consisting of obesity, insulin resistance, type 2 diabetes, hyperglycemia, hypercholesterolemia, atherosclerosis, and other related disease.

46. The method of claim 43, further comprising identifying a subject in need of prevention or treatment for ER stress-related diseases or conditions.

47. The method of claim 43, further comprising the step of obtaining the compound of Formula I.

48. The method of claim 43, wherein the subject is a mammal.

49. The method of claim 43, in which the subject is a human.

50. A pharmaceutical composition, comprising an effective amount of a compound represented by the formula: 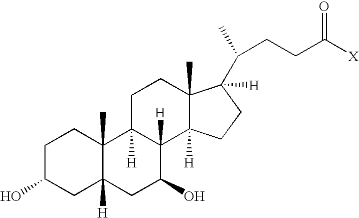
in which X is one of the following moieties: 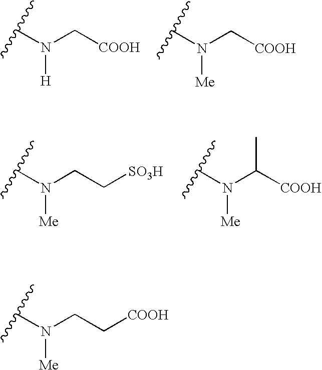
and pharmaceutically acceptable esters, salts, and prodrugs thereof;
and a pharmaceutically acceptable carrier.

51. The pharmaceutical composition of claim 50, wherein said effective amount is effective to treat an ER-stress associated state.

52. The pharmaceutical composition of claim 50, wherein said ER-stress associated state is a disorder selected from the group consisting of obesity insulin resistance, type 2 diabetes hyperglycemia, hypercholesterolemia, atherosclerosis and related diseases.

53. A packaged formulation comprising a pharmaceutical composition of claim 50, and instructions for use in the treatment of an ER-stress associated state.

54. The packaged formulation of claim 53, wherein said compound is present in an amount effective to treat the ER-stress associated state.

55. The packaged formulation of claim 53, wherein said ER-stress associated state is a disorder selected from the group consisting of obesity insulin resistance, type 2 diabetes hyperglycemia, hypercholesterolemia, atherosclerosis and related diseases.

56-58. (canceled)

59. A method for treating or preventing a condition related to ER-stress in a subject in need thereof, comprising administering to said subject an effective amount of a compound of claim 27 such that said condition related to ER-stress is treated or prevented.

60. A method for treating or preventing a condition related to ER-stress in a subject in need thereof, comprising administering to said subject an effective amount of a compound selected from the group consisting of: 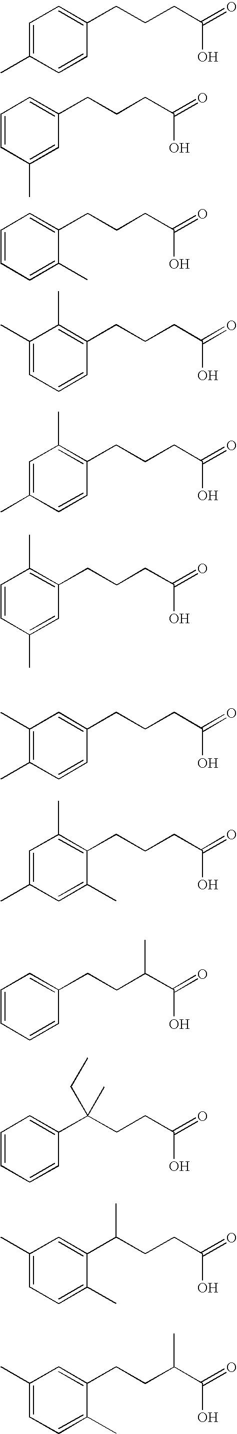 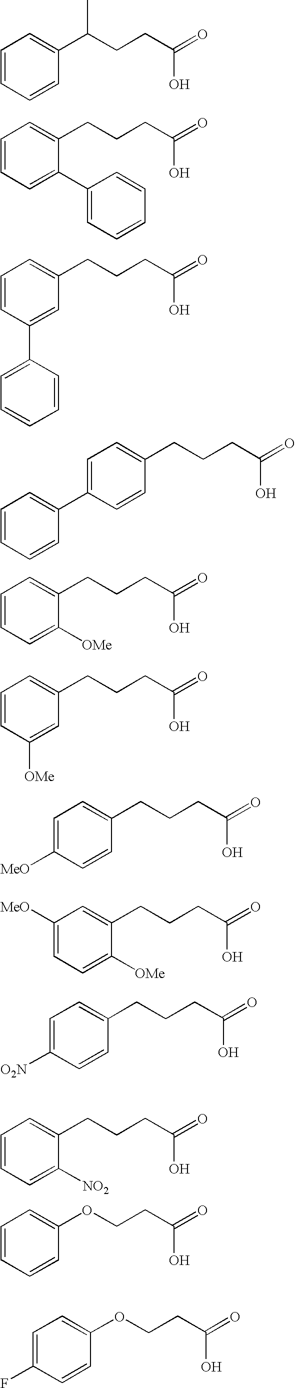 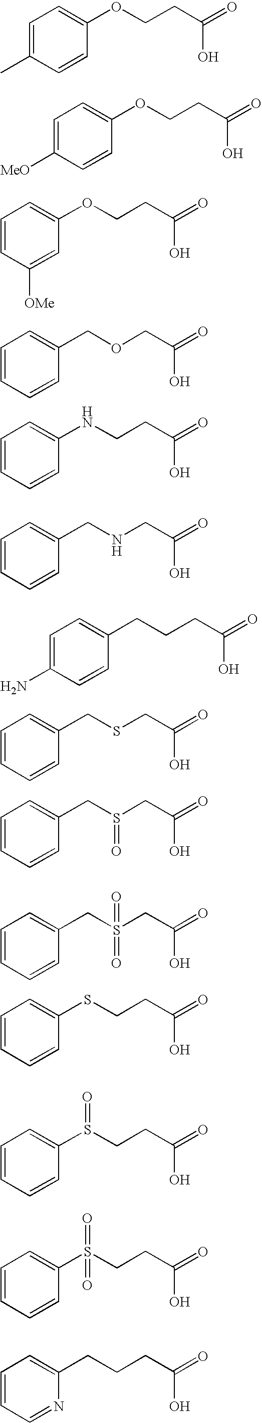 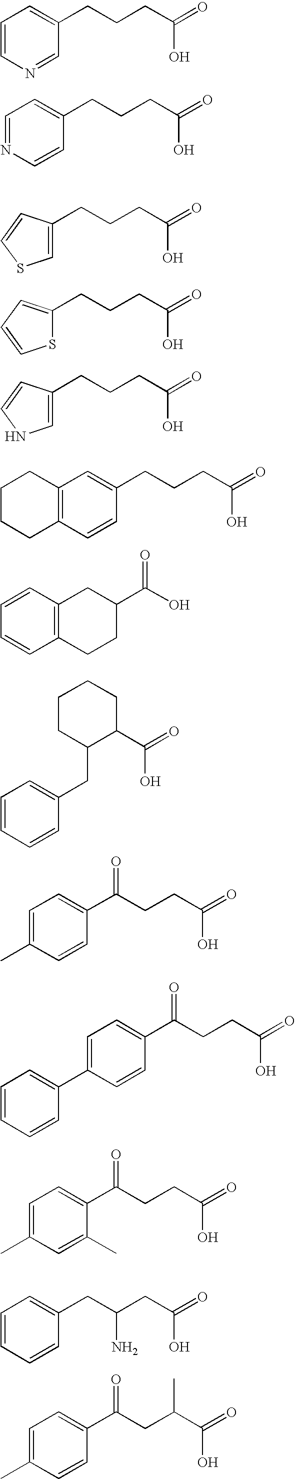 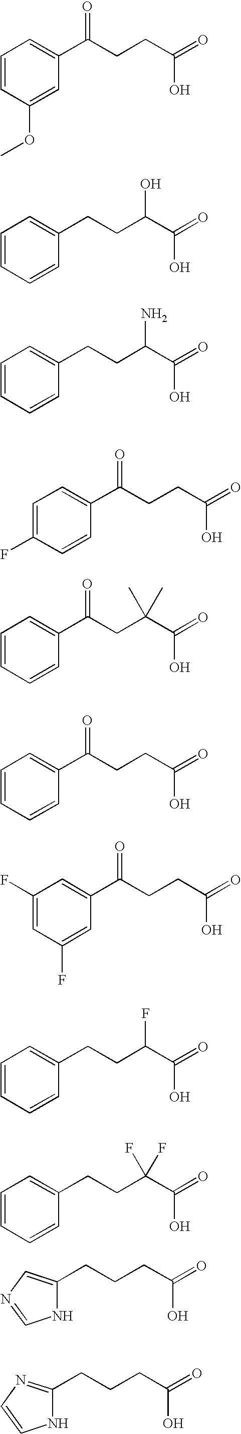 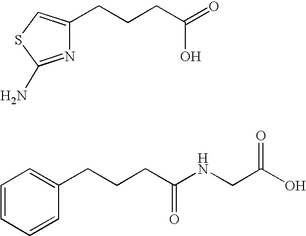
and pharmaceutically acceptable esters, salts, and prodrugs thereof, such that said condition related to ER-stress is treated for prevented.

61-65. (canceled)

66. A pharmaceutical composition, comprising an effective amount of a compound selected from the group consisting of: 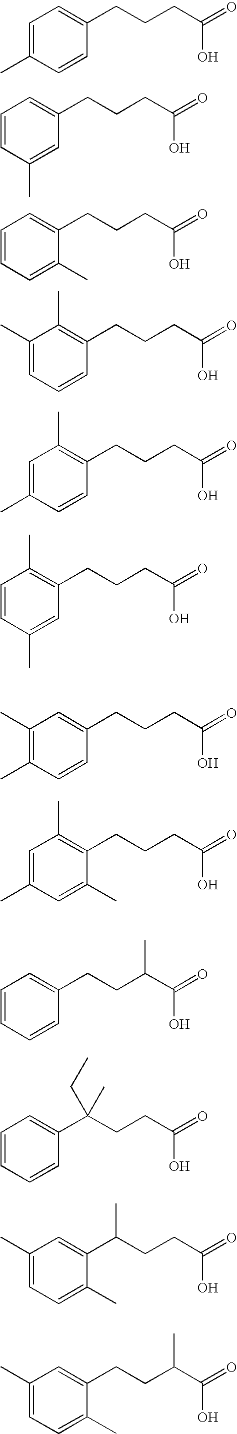 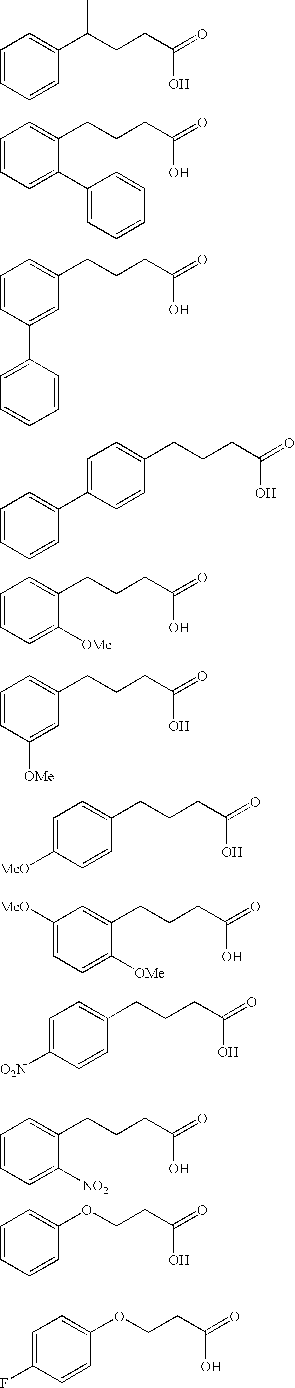 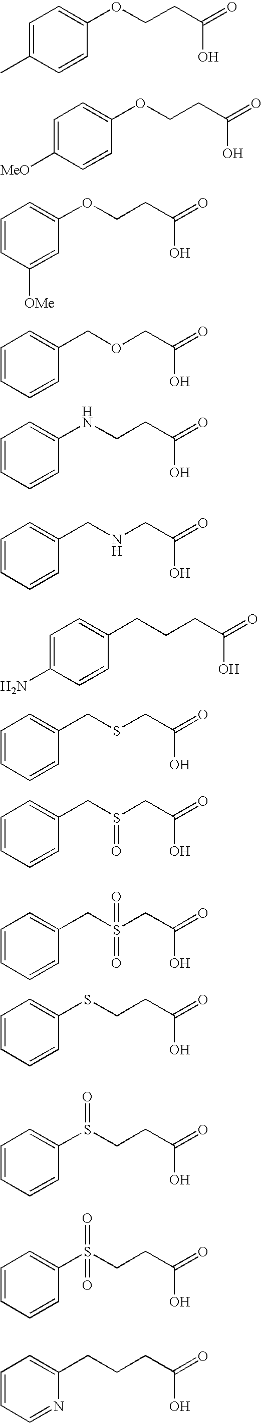 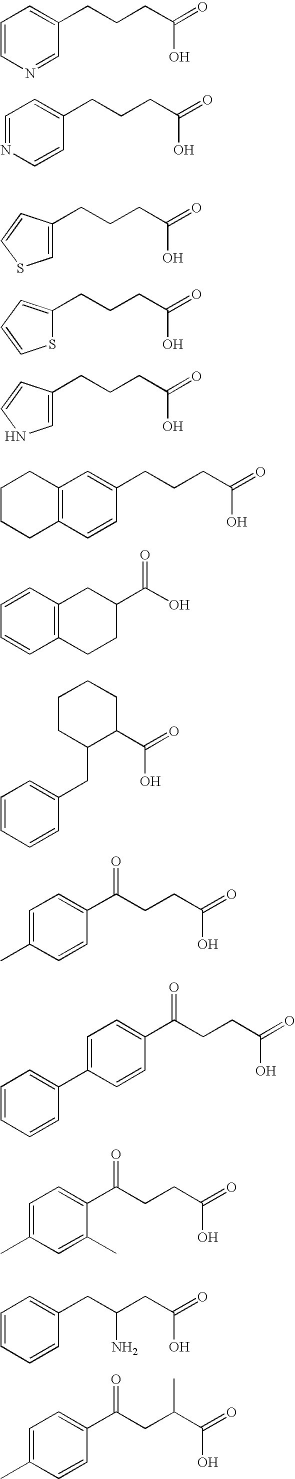 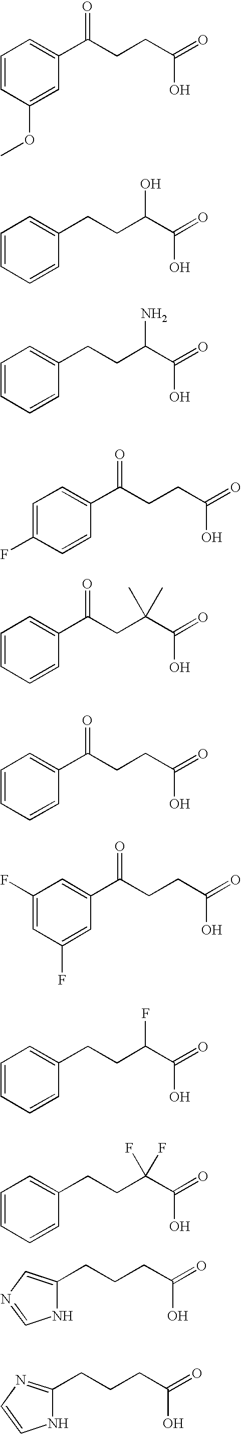 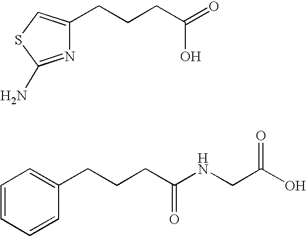
and pharmaceutically acceptable esters, salts, and prodrugs thereof;
and a pharmaceutically acceptable carrier.

67. (canceled)

68. (canceled)

69. A packaged formulation comprising a pharmaceutical composition of claim 66, and instructions for use in the treatment of an ER-stress associated state.

70. (canceled)

71. (canceled)

72. The method of claim 43, wherein the condition related to ER-stress is hypercholesterolemia.

73. The method of claim 44, wherein the condition related to ER-stress is hypercholesterolemia.

74-77. (canceled)

78. The method of claim 43, wherein the condition related to ER-stress is atherosclerosis.

79. The method of claim 44, wherein the condition related to ER-stress is atherosclerosis.

80-83. (canceled)

84. A packaged formulation comprising a pharmaceutical composition comprising a compound represented by Formula I: 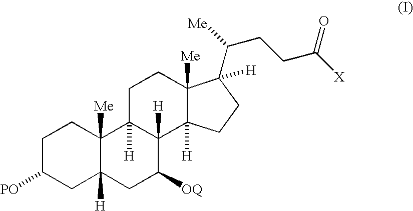
in which
P and Q are independently for each occurrence H, lower alkyl, PO₃R₂, SO₃R or COR;
X is OR, NR₂ or NR—B-D;
B is alkylene, substituted alkylene, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″, or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R is H, lower alkyl, aryl, or heterocyclyl;
R₁-R₄ are independently for each occurrence H, lower alkyl, fluoro or haloalkyl;
R₅ is H, lower alkyl, or substituted or unsubstituted aryl, or substituted or unsubstituted heteroaryl;
m is 1-3; and
n is 0-3;
and pharmaceutically acceptable esters, salts, and prodrugs thereof;
and instructions for use in the treatment of hypercholesterolemia;
provided that the compound is not a compound represented by the formula: 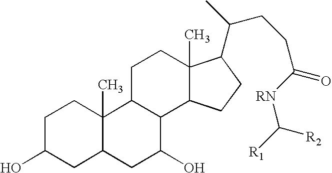
wherein
R is —H or C₁-C₄ alkyl;
R₁ is CH₂—SO₃R₃ and R₂ is —H; or R₁ is —COOH and R₂ is —CH₂—CH₂—CONH₂, —CH₂—CONH₂, —CH₂—CH₂—SCH₃ or —CH₂—S—CH₂—COOH; and
R₃ is —H or a basic amino acid; or a pharmaceutically acceptable salt thereof.

85. The packaged formulation of claim 84, wherein said compound is present in an amount effective to treat hypercholesterolemia.

86. A packaged formulation comprising a pharmaceutical composition comprising a compound represented by Formula I: 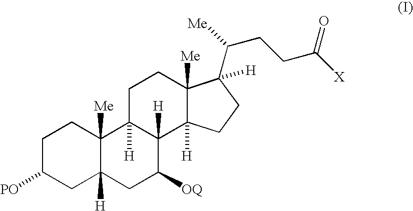
in which
P and Q are independently for each occurrence H, lower alkyl, PO₃R₂, SO₃R or COR;
X is OR, NR₂ or NR—B-D;
B is alkylene, substituted alkylene, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″, or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R is H, lower alkyl, aryl, or heterocyclyl;
R₁-R₄ are independently for each occurrence H, lower alkyl, fluoro or haloalkyl;
R₅ is H, lower alkyl, or substituted or unsubstituted aryl, or substituted or unsubstituted heteroaryl;
m is 1-3; and
n is 0-3;
and pharmaceutically acceptable esters, salts, and prodrugs thereof;
and instructions for use in the treatment of atherosclerosis;
provided that the compound is not a compound represented by the formula: 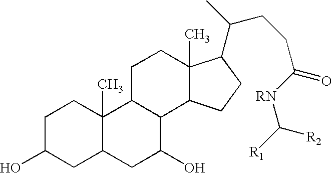
wherein
R is —H or C₁-C₄ alkyl;
R₁ is CH₂—SO₃R₃ and R₂ is —H; or R₁ is —COOH and R₂ is —CH₂—CH₂—CONH₂, —CH₂—CONH₂, —CH₂—CH₂—SCH₃ or —CH₂—S—CH₂—COOH; and
R₃ is —H or a basic amino acid; or a pharmaceutically acceptable salt thereof.

87. The packaged formulation of claim 86, wherein said compound is present in an amount effective to treat atherosclerosis.

88. (canceled)

89. (canceled)

90. A method for treating or preventing hypercholesterolemia in a subject in need thereof, comprising administering to said subject an effective amount of a compound represented by the formula: 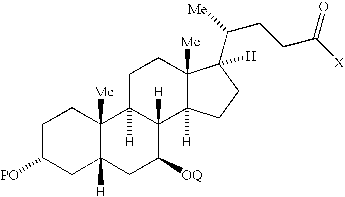
in which X is: 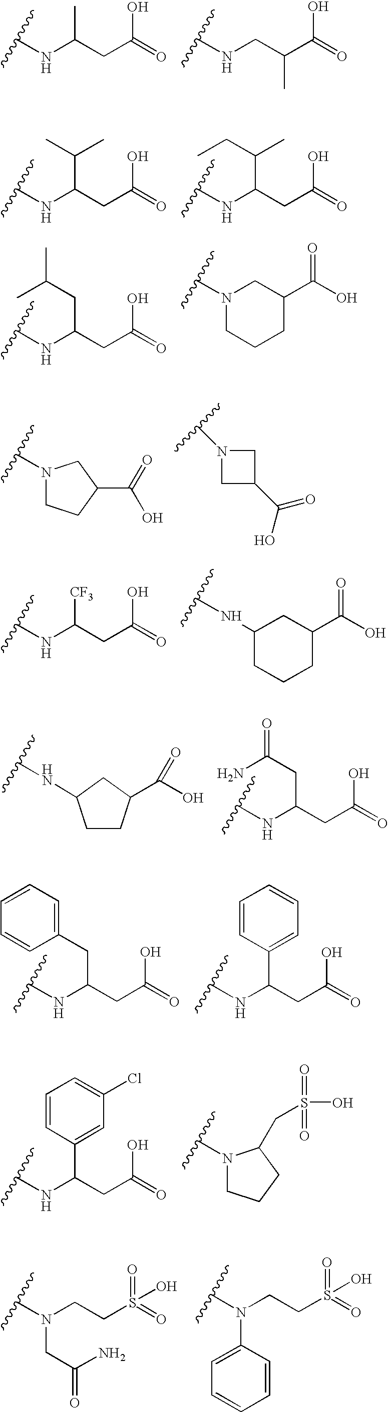 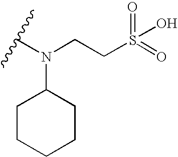
and pharmaceutically acceptable esters, salts, and prodrugs thereof;
such that said hypercholesterolemia is treated or prevented.

91-96. (canceled)

97. A method for treating or preventing atherosclerosis in a subject in need thereof, comprising administering to said subject an effective amount of a compound represented by the formula: 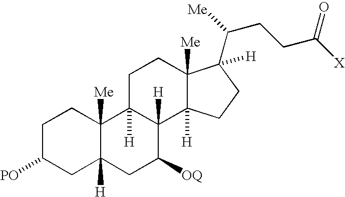
in which X is: 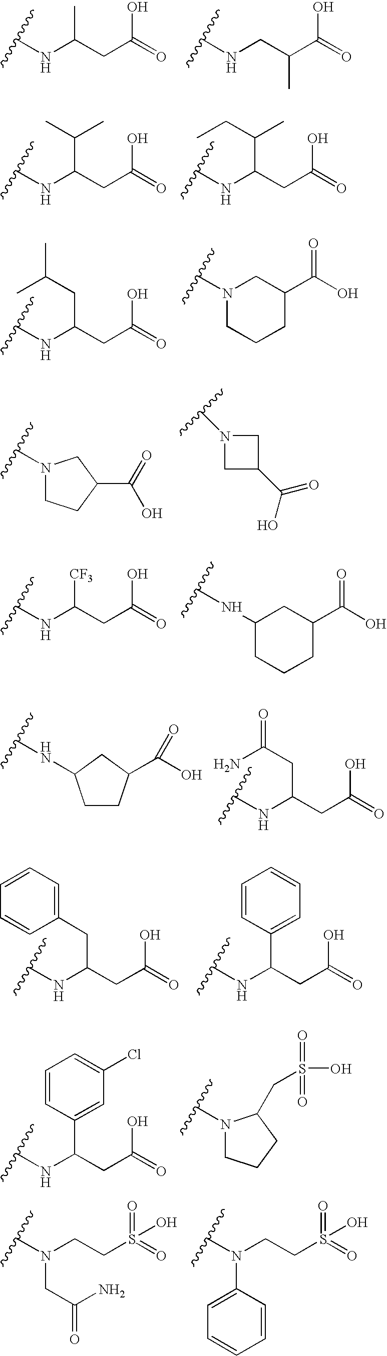 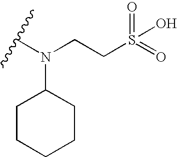
and pharmaceutically acceptable esters, salts, and prodrugs thereof;
such that said atherosclerosis is treated or prevented.

98-101. (canceled)

102. A packaged formulation comprising a pharmaceutical composition comprising a compound represented by Formula I: 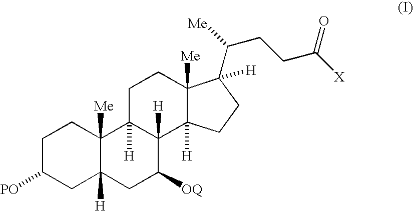
in which
P and Q are independently for each occurrence H, lower alkyl, PO₃R₂, SO₃R or COR;
X is OR, NRₐR_{b} or NR—B-D;
B is alkylene, substituted alkylene, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″, or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R is H, lower alkyl, aryl, or heterocyclyl;
R₁-R₄ are independently for each occurrence H, lower alkyl, fluoro or haloalkyl;
R₅ is H, lower alkyl, or substituted or unsubstituted aryl, or substituted or unsubstituted heteroaryl;
m is 1-3; and
n is 0-3;
and pharmaceutically acceptable esters, salts, and prodrugs thereof;
and instructions for use in the treatment of hypercholesterolemia;
provided that the compound is not a compound represented by the formula: 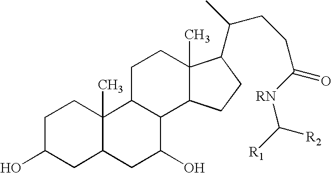
wherein
R is —H or C₁-C₄ alkyl;
R₁ is CH₂—SO₃R₃ and R₂ is —H; or R₁ is —COOH and R₂ is —CH₂—CH₂—CONH₂, —CH₂—CONH₂, —CH₂—CH₂—SCH₃ or —CH₂—S—CH₂—COOH; and
R₃ is —H or a basic amino acid; or a pharmaceutically acceptable salt thereof.

103. The packaged formulation of claim 102, wherein said compound is present in an amount effective to treat hypercholesterolemia.

104. A packaged formulation comprising a pharmaceutical composition comprising a compound represented by Formula I: 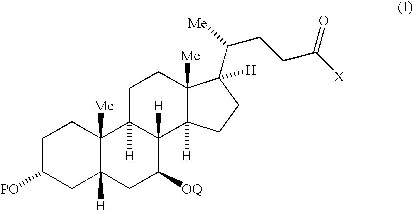
in which
P and Q are independently for each occurrence H, lower alkyl, PO₃R₂, SO₃R or COR;
X is OR, NRₐR_{b} or NR—B-D;
B is alkylene, substituted alkylene, or —(CR₁R₂)ₙ-E-(CR₃R₄)ₘ—;
D is CO₂R, CONRₐR_{b}, SO₃R, SO₂NRₐR_{b}, tetrazolyl, —B(OR)(OR″), —P(O)R′OR″, or PO₃R′R″;
E is O, S, SO, SO₂, —SO₂N(R₅)—, —N(R₅)SO₂—, NR₅, —C(O)O—, —O(O)C—, —CONR₅—, —NR₅CO—, or is absent;
R, R′ and R″ are independently for each occurrence H or lower alkyl;
Rₐ and R_{b} are independently for each occurrence H, OH, lower alkyl, substituted or unsubstituted aryl, or substituted or unsubstituted heteroaryl, provided that Rₐ and R_{b} are not both OH;
R is H, lower alkyl, aryl, or heterocyclyl;
R₁-R₄ are independently for each occurrence H, lower alkyl, fluoro or haloalkyl;
R₅ is H, lower alkyl, or substituted or unsubstituted aryl, or substituted or unsubstituted heteroaryl;
m is 1-3; and
n is 0-3;
and pharmaceutically acceptable esters, salts, and prodrugs thereof;
and instructions for use in the treatment of atherosclerosis;
provided that the compound is not a compound represented by the formula: 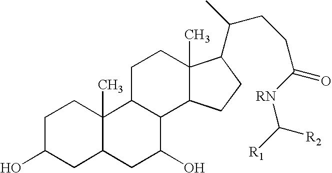
wherein
R is —H or C₁-C₄ alkyl;
R₁ is CH₂—SO₃R₃ and R₂ is —H; or R₁ is —COOH and R₂ is —CH₂—CH₂—CONH₂, —CH₂—CONH₂, —CH₂—CH₂—SCH₃ or —CH₂—S—CH₂—COOH; and
R₃ is —H or a basic amino acid; or a pharmaceutically acceptable salt thereof.

105. The packaged formulation of claim 104, wherein said compound is present in an amount effective to treat atherosclerosis.

106. The method of claim 31, wherein the condition related to ER-stress is hypercholesterolemia.

107. A method for treating or preventing hypercholesterolemia in a subject in need thereof, comprising administering to said subject an effective amount of a compound of claim 25;
such that said hypercholesterolemia is treated or prevented.

108. The method of claim 59 wherein the condition related to ER-stress is hypercholesterolemia.

109. The method of claim 60 wherein the condition related to ER-stress is hypercholesterolemia.

110-113. (canceled)

114. The method of claim 31, wherein the condition related to ER-stress is atherosclerosis.

115. A method for treating or preventing atherosclerosis in a subject in need thereof, comprising administering to said subject an effective amount of a compound of claim 25;
such that said atherosclerosis is treated or prevented.

116. The method of claim 59, wherein the condition related to ER-stress is atherosclerosis.

117. The method of claim 60 wherein the condition related to ER-stress is atherosclerosis.

118-125. (canceled)

126. A method for treating or preventing a condition related to ER-stress in a subject in need thereof, comprising administering to said subject an effective amount of a compound represented by Formula IV: 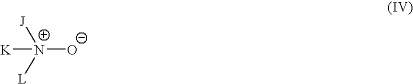
wherein
J, K, and L are independently for each occurrence alkyl, substituted alkyl, or —(CR₁R₂)ₙ-E-(CR₃R₄)ₘ—X;
X is H or CH₃;
R is independently for each occurrence H, lower alkyl, aryl, heterocyclyl;
R₁-R₄ are independently for each occurrence H, lower alkyl, fluoro or haloalkyl;
R₅ is independently for each occurrence H, lower alkyl, aryl, heterocyclyl;
m is independently for each occurrence 0-3;
n is independently for each occurrence 1-3;
E is O, S, SO, SO₂, —SO₂N(R₅)—, —N(R₅)SO₂—, NR₅, —C(O)O—, —O(O)C—, —CON(R₅)—, —N(R₅)CO—, or is absent;
provided that when m=0, E is selected from O, NR₅R₆, CONR₅R₆, SO₂NR₅R₆; and
R₆═H, lower alkyl, aryl, heterocyclyl;
and pharmaceutically acceptable esters, salts, and prodrugs thereof;
such that said subject is treated for said ER-stress associated state;
provided that the compound is not a compound represented by the formula: 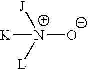
wherein
R₁, R₂, and R₃ are independently hydrogen, halogen, or lower C₁-C₆ alkyl;
or a pharmaceutically-acceptable salt thereof; or a mixture thereof.

127-132. (canceled)

133. A pharmaceutical composition, comprising an effective amount a compound recited in the method of claim 126.

134-138. (canceled)

139. The method of claim 136, wherein the condition related to ER-stress is hypercholesterolemia.

140-145. (canceled)

146. The method of claim 136, wherein the condition related to ER-stress is atherosclerosis.

147-152. (canceled)

153. A packaged formulation comprising a pharmaceutical composition comprising a compound recited in the method of claim 139;
and instructions for use in the treatment of hypercholesterolemia.

154. (canceled)

155. A packaged formulation comprising a pharmaceutical composition comprising a compound recited in the method of claim 146
and instructions for use in the treatment of atherosclerosis.

156-170. (canceled)

171. A method for treating or preventing hypercholesterolemia in a subject in need thereof, comprising administering to said subject an effective amount of a compound selected from the group consisting of: 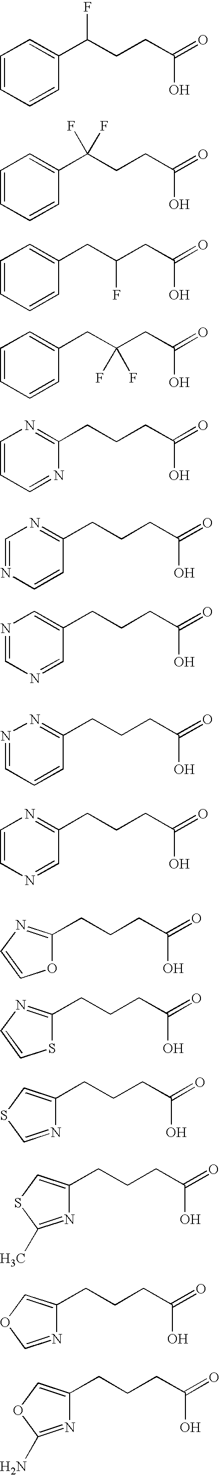 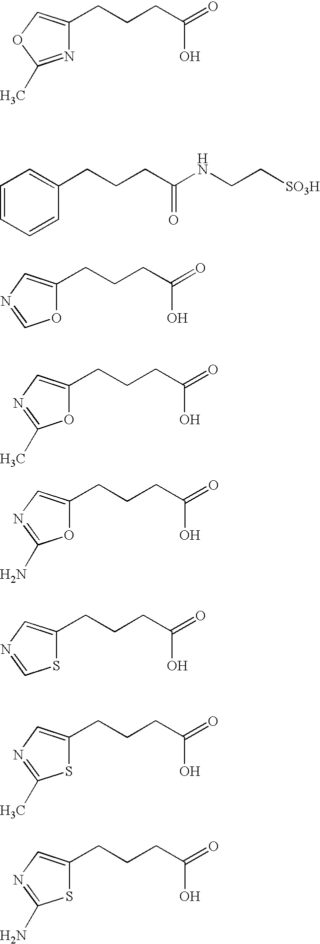
and pharmaceutically acceptable esters, salts, and prodrugs thereof;
such that said hypercholesterolemia is treated or prevented.

172-189. (canceled)

190. A method for treating or preventing atherosclerosis in a subject in need thereof, comprising administering to said subject an effective amount of a compound selected from the group consisting of: 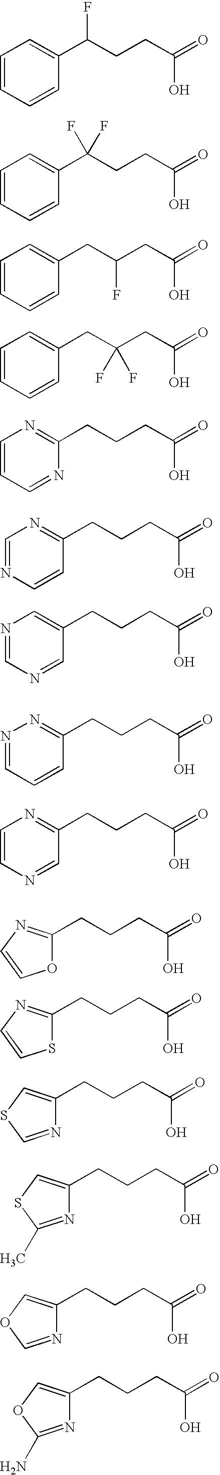 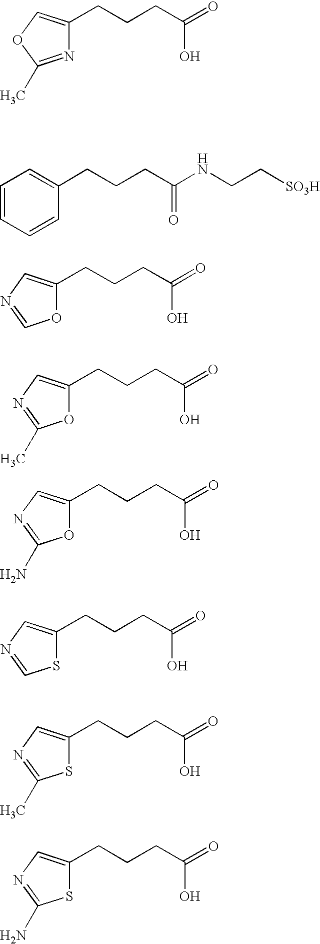
and pharmaceutically acceptable esters, salts, and prodrugs thereof;
such that said atherosclerosis is treated or prevented.

191-194. (canceled)
